SEQUENCE LISTING (47 sequences)

SEQ ID NO: 1 - DNA, 26 bp, Artificial
  chemically synthesized
gcgcacatgtcttacagtatcactac

SEQ ID NO: 2 - DNA, 46 bp, Artificial
  chemically synthesized
tggtcctgcaactgccatggacagtgccggctgaggtagtcaagat

SEQ ID NO: 3 - DNA, 22 bp, Artificial
  chemically synthesized
cggataacaatttcacacagga

SEQ ID NO: 4 - DNA, 27 bp, Artificial
  chemically synthesized
ccacatgtatacaatcaactctccgag

SEQ ID NO: 5 - DNA, 21 bp, Artificial
  chemically synthesized
cactgtccatggctgtggtcc

SEQ ID NO: 6 - DNA, 48 bp, Artificial
  chemically synthesized
cttgtctggaccacagccatggacagtgccggcactccggctacttag

SEQ ID NO: 7 - DNA, 26 bp, Artificial
  chemically synthesized
aaacatgattacgccaagcttgcatg

SEQ ID NO: 8 - DNA, 498 bp, Artificial
  Peptide acceptor vector pLSB2110 open reading frame
atgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaactgccatggacagtgccggctga

SEQ ID NO: 9 - DNA, 495 bp, Artificial
  Peptide acceptor vector pLSB1806 open reading frame
atgtatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacagccatggacagtgccggcactccggctacttag

SEQ ID NO: 10 - protein, 20 aa, Artificial
  20 amino acid DJ5 epitope from human
VHQANPRGSAGPCCTPTKMS

SEQ ID NO: 11 - protein, 12 aa, Artificial
  12 amino acid DJ5-derived epitope from human
VHQANPRGSAGP

SEQ ID NO: 12 - DNA, 65 bp, Artificial
  Forward oligonucleotide for 20 amino acid DJ5 peptide cloning, chemically synthesized
catggttcatcaagctaatccaagaggatctgctggaccatgttgtactccaactaagatgtctg

SEQ ID NO: 13 - DNA, 65 bp, Artificial
  Reverse oligonucleotide for 20 amino acid DJ5 peptide cloning, chemically synthesized
ccggcagacatcttagttggagtacaacatggtccagcagatcctcttggattagcttgatgaac

SEQ ID NO: 14 - DNA, 549 bp, Artificial
  ORF for vector pLSB2655
atgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctgccatggttcatcaagctaatccaagaggatctgctggaccatgttgtactccaactaagatgtctgccggccctgcaacttga

SEQ ID NO: 15 - DNA, 552 bp, Artificial
  ORF for vector pLSB2656
atgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaactgccatggttcatcaagctaatccaagaggatctgctggaccatgttgtactccaactaagatgtctgccggctga

SEQ ID NO: 16 - DNA, 546 bp, Artificial
  ORF for vector pLSB2657
atggttcatcaagctaatccaagaggatctgctggaccatgttgtactccaactaagatgtctgccggctcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttga

SEQ ID NO: 17 - DNA, 561 bp, Artificial
  ORF for vector pLSB2658
atgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctggatctcccatggttcatcaagctaatccaagaggatctgctggaccatgttgtactccaactaagatgtctgccggcccttctggagactttaaggtatacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttga

SEQ ID NO: 18 - DNA, 549 bp, Artificial
  ORF for vector pLSB2659
atgtatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacagccatggttcatcaagctaatccaagaggatctgctggaccatgttgtactccaactaagatgtctgccggcactccggctacttag

SEQ ID NO: 19 - protein, 182 aa, Artificial
  Coat protein amino acid sequence obtained from pLSB2655
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSAMVHQANPRGSAGPCCTPTKMSAGPAT

SEQ ID NO: 20 - protein, 182 aa, Artificial
  Coat protein amino acid sequence obtained from pLSB2656
SYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPATAMVHQANPRGSAGPCCTPTKMSAG

SEQ ID NO: 21 - protein, 181 aa, Artificial
  Coat protein amino acid sequence obtained from pLSB2657
MVHQANPRGSAGPCCTPTKMSAGSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 22 - protein, 186 aa, Artificial
  Coat protein amino acid sequence obtained from pLSB2658
MSYSITTPSQFVFLSSAWADPIELINLCTNALGNQFQTQQARTVVQRQFSEVWKPSPQVTVRFPGSPMVHQANPRGSAGPCCTPTKMSAGPSGDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 23 - protein, 182 aa, Artificial
  Coat protein amino acid sequence obtained from pLSB2659
MYTINSPSQFVYLSSAYADPVQLINLCTNALGNQFQTQQARTTVQQQFADAWKPVPSMTVRFPASDFYVYRYNSTLDPLITALLNSFDTRNRIIEVDNQPAPNTTEIVNATQRVDDATVAIRASINNLANELVRGTGMFNQASFETASGLVWTTAMVHQANPRGSAGPCCTPTKMSAGTPAT

SEQ ID NO: 24 - DNA, 41 bp, Artificial
  chemically synthesized
catggttcatcaagctaatccaagaggatctgctggaccag

SEQ ID NO: 25 - DNA, 41 bp, Artificial
  chemically synthesized
ccggctggtccagcagatcctcttggattagcttgatgaac

SEQ ID NO: 26 - DNA, 522 bp, Artificial
  ORF for vector pLSB2663
atggttcatcaagctaatccaagaggatctgctggaccagccggctcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttga

SEQ ID NO: 27 - DNA, 525 bp, Artificial
  ORF for vector pLSB2664
atgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctgccatggttcatcaagctaatccaagaggatctgctggaccagccggccctgcaacttga

SEQ ID NO: 28 - protein, 173 aa, Artificial
  Coat protein amino acid sequence obtained from pLSB2663
MVHQANPRGSAGPAGSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 29 - protein, 174 aa, Artificial
  Coat protein amino acid sequence obtained from pLSB2664
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSAMVHQANPRGSAGPAGPAT

SEQ ID NO: 30 - protein, 159 aa, Tobacco mosaic virus strain U1
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 31 - protein, 159 aa, Tobacco mosaic virus strain U5
MPYTINSPSQFVYLSSAYADPVQLINLCTNALGNQFQTQQARTTVQQQFADAWKPVPSMTVRFPASDFYVYRYNSTLDPLITALLNSFDTRNRIIEVDNQPAPNTTEIVNATQRVDDATVAIRASINNLANELVRGTGMFNQASFETASGLVWTTTPAT

SEQ ID NO: 32 - protein, 12 aa, Artificial
  coat protein fusion amino acid sequence (p15E DE)
DEKSPWFTTLAG

SEQ ID NO: 33 - protein, 14 aa, Artificial
  coat protein fusion amino acid sequence (HPV 6/11 L2)
GLIEESAIINAGAP

SEQ ID NO: 34 - protein, 25 aa, Artificial
  coat protein fusion amino acid sequence (HPV 16 L1 I23)
GQPLGVGISGHPLLNKLDDTEGPAT

SEQ ID NO: 35 - protein, 16 aa, Artificial
  coat protein fusion amino acid sequence (IL1)
AMVQGEESNDKAGPAT

SEQ ID NO: 36 - protein, 19 aa, Artificial
  CRPV L2.1 epitope
GVGPLDIVPEVADPGGPTL

SEQ ID NO: 37 - protein, 14 aa, Artificial
  HPV 6/11 L2 epitope
GLIEESAIINAGAP

SEQ ID NO: 38 - protein, 19 aa, Artificial
  ROPV L2.2 epitope
GPAGSSIVPLEEYPAEIPT

SEQ ID NO: 39 - protein, 19 aa, Artificial
  ROPV L2.1 epitope
GVGPLEVIPEAVDPAGSSI

SEQ ID NO: 40 - protein, 7 aa, Artificial
  derived from the gp41 protein of HIV
ELDKWAS

SEQ ID NO: 41 - protein, 14 aa, Artificial
  coat protein fusion HPV 6/11
GLIEESAIINAGAP

SEQ ID NO: 42 - protein, 12 aa, Artificial
  coat protein fusion IL1 b
AMVQGEESNDKA

SEQ ID NO: 43 - protein, 12 aa, Artificial
  12 amino acid goat epitope G5
VHQANPKGSAGP

SEQ ID NO: 44 - protein, 24 aa, Artificial
  20 amino acid human epitope DJ5 plus last 4 amino acids of TMV U1 Coat Protein
VHQANPRGSAGPCCTPTKMSGPAT

SEQ ID NO: 45 - protein, 24 aa, Artificial
  20 amino acid goat epitope G5 plus last 4 amino acids of TMV U1 Coat Protein
VHQANPKGSAGPCCTPTKMSGPAT

SEQ ID NO: 46 - protein, 4 aa, Tobacco mosaic virus strain U1
GPAT

SEQ ID NO: 47 - protein, 4 aa, Tobacco mosaic virus strain U5
TPAT

(19) United States
(12) Patent Application Publication
(10) Pub. No.: US 2006/0281075 A1
(43) Pub. Date: Dec. 14, 2006

(54) Purification of viruses, proteins and nucleic acids

(75) Inventors: Mark L. Smith, Davis, CA (US); Amanda Lasnik, Sacramento, CA (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA (US)

(21) Appl. No.: 11/317,282

(22) Filed: Dec. 22, 2005

Related U.S. Application Data
(60) Provisional application No. 60/638,283, filed on Dec. 22, 2004.
(60) Provisional application No. 60/638,284, filed on Dec. 22, 2004.

(51) Int. Cl. C12Q 1/70; C12Q 1/68; C12N 7/02

(52) U.S. Cl. 435/5; 435/6; 435/239

(57) ABSTRACT

Viruses, virus-like particles and protein and nucleic acid components are extracted from biological material using high ionic strength, activated carbon and changes in pH. The purified compositions are stored in similar or different conditions preferably with a higher pH.

### CROSS REFERENCE TO RELATED APPLICATIONS

This application claims priority to U.S. Provisional Applications Ser. No. 60/638,283 and 60/638,284, both filed Dec. 22, 2004, hereby incorporated by reference.

### 1. FIELD OF THE INVENTION

This application relates to methods for extracting and purifying viruses, virus like particles from biological sources.

### 2. BACKGROUND INFORMATION

Recombinant proteins have been produced using virus-derived vectors based on the rod-shaped plant virus Tobacco Mosaic Virus (TMV) (Pogue, G. P., J. A. Lindbo, et al. (2002). Making an ally from an enemy: Plant virology and the new agriculture. Ann Rev Phytopathol 40: 45-74). TMV has a plus sense single stranded RNA genome of approximately 6400 nucleotides. The viral proteins involved in RNA replication are directly transcribed from the genomic RNA, whereas expression of internal genes is through the production of subgenomic RNAs. The production of subgenomic RNAs is controlled by sequences in the TMV genome, which function as subgenomic promoters. The coat protein (CP) is translated from a subgenomic RNA and is the most abundant protein and RNA produced in the infected cell (FIG. 1). In a TMV infected plant there are several milligrams of CP produced per gram of infected tissue. Such expression vectors take advantage of both the strength and duration of this promoter's activity to reprogram the translational priorities of the plant host cells so that virus-encoded proteins are synthesized at high levels, similar to the TMV CP.

This expression system has been used to produce many important recombinant proteins and antigens including important protein antigens that have been used as effective immunogens (Pogue, G. P., J. A. Lindbo, et al. (2002). Making an ally from an enemy: Plant virology and the new agriculture. Ann Rev Phytopathol 40: 45-74). Soluble proteins produced in plants using the TMV vector system are extracted from the plant by tissue homogenization and clarification methods and purified using standard chromatographic separations. This system has been demonstrated as safe and environmentally-friendly in outdoor field tests from 1991 through 2004 and 16 products produced by a tobamovirus expression system have been shown to be safe in human clinical trials.

Two distinct methods allow expression of foreign proteins or peptides by: 1) Independent gene expression: by adding a foreign gene for expression in place of the virus coat protein so it will be expressed from the endogenous virus coat protein promoter. A second coat protein promoter of lesser transcriptional activity and non-identity in sequence is placed downstream of the heterologous coding region and a virus coat protein gene is then added. This encodes a third subgenomic RNA allowing the virus vector to express all requisite genes for virus replication and systemic movement in addition to the heterologous gene intended for overexpression. 2) Display of immunogenic peptides on the surface of virus particles. The TMV virion is a rigid rod of ˜18 nm diameter and 300 nm length. The structure of the virion and coat protein has been determined by X-ray diffraction revealing a structure of approximately 2,130 coat protein subunits arranged in a right-handed helix encapsidating the genomic RNA, with 16.3 subunits per turn. Many different peptides have been fused to the TMV coat protein N-terminus, C-terminus or loop region, such that the peptides are displayed at unprecedented density. The resulting TMV virions are readily purified from plants and have proven to often produce protective immune responses, in experimental mammalian systems (Pogue, G. P., J. A. Lindbo, et al. (2002). Making an ally from an enemy: Plant virology and the new agriculture. Ann Rev Phytopathol 40: 45-74).

Numerous procedures have been described for the purification of TMV virus coat protein fusions. For examples Garger et al. (U.S. Pat. Nos. 6,033,895, 6,037,456, 6,303,779 and 6,740,740 and Pogue et al. (U.S. Pat. No. 6,730,306) disclose methods based on the pH adjustment and heat treatment of the homogenate “green juice” obtained following extraction of the infected tissue. Pogue et al. also disclose a procedure based on the use of polyethyleneimine (PEI) to aid in the separation of the plant host proteins and the recombinant TMV. The published literature also contain examples involving the use of activated carbon during the purification of wild-type TMV and a number of other rod shaped and icosahedral plant viruses to remove natural plant components during virus purification. In early work by Price (Price, W. C., Purification and crystallization of southern bean mosaic virus. Am. J. Botany, 1946. 33: p. 45-54), a dark pigment component, present in concentrated preparations of southern bean mosaic virus (purified from plants grown at high temperature in the summer), was removed by adding activated charcoal to the virus suspension and then filtering through Celite to remove the charcoal. A similar procedure was employed in the purification of lettuce necrotic yellow virus (Lean, G. D. and R. I. B. Francki, Purification of lettuce necrotic yellow virus by column chromatography on calcium phosphate gel. Virology, 1967. 31: p. 585-591). The plant tissue homogenate, obtained by extracting in 1.5 volumes of 200 mm dibasic sodium phosphate (final pH of 7.0) was clarified by a low speed centrifugation and the supernatant contacted with activated carbon at 5% w/v for 30 seconds, with activated carbon removal by filtration through Celite. The Celite filtration also removed the majority of the green pigment.

Activated carbon was employed in the purification of potato virus X (PVX), a flexous rod shaped virus (Corbett, M. K., Purification of potato virus X without aggregation. Virology, 1961. 15: p. 8-15). Briefly, the plant extract was initially subjected to a low speed centrifugation for 15 minutes, and the clarified sap then contacted with 10% w/v activated carbon for 30 minutes, with filtration employed to remove the carbon following treatment. Corbett noted that green pigment remained associated with the sap following activated carbon treatment. A procedure similar to that employed with PVX was an effective component in the purification of-tobacco ringspot virus; the clarified sap was again contacted with 10% w/v activated carbon for 30 minutes (Corbett, M. K. and D. A. Roberts, A rapid method for purifying tobacco ringspot virus and its morphology as determined by electron microscopy and negative staining. Phytopathology, 1962. 52: p. 902-905). In one protocol employed in the purification of TMV, the plant extract was maintained at pH 7.5, and contacted with activated carbon at 5% w/v for 20-30 seconds, followed by the addition of 5% w/v diatomaceous earth and agitation for a further 20-30 seconds. The charcoal and diatomaceous earth solids were then removed by filtration. By this procedure the colored components of the extract were retained in the filter cake and the virus was present in the filtrate (Steere, R. L., Tobacco mosaic virus: purifying and sorting associated particles according to length. Science, 1963. 140: p. 1089-1090). Von Wechmar and van Regenmortel (1970) also employed activated carbon at 5% w/v together with diatomaceous earth (5% w/v) with a contact time of 30 seconds in the purification of TMV. The solids were subsequently removed by filtration, prior to the precipitation of the virus by polyethylene glycol. Activated carbon treatment has also been combined with organic extraction. For example, Timian and Savage (Timian, R. G. and S. M. Savage, Purification of barley stripe mosaic virus with chloroform and charcoal. Phytopathology, 1966. 56: p.1233-1235), employed a combination of chloroform and activated carbon treatment to purify barley stripe mosaic virus: (BSMV). Plant material was homogenized in the absence of buffer and the sap centrifuged for 10 minutes at 4,520×g. The supernatant was chloroform treated and after separation and removal of the chloroform by an additional centrifugation and decanting, activated carbon (7.5% w/v) was added and contacted for 15 minutes prior to removal by centrifugation. The chloroform treatment removed the green pigments and the brownish yellow pigments were adsorbed by the activated carbon treatment.

Finally, the method of activated carbon removal was shown to influence purified virus recovery (Galvez, G. E., Loss of virus by filtration through charcoal. Virology, 1964. 23: p.307-312). Using plant sap infected with both rod-shaped (TMV) and icosahedral viruses (BMV & southern bean mosaic virus, SBMV) a number of parameters relating to activated carbon treatment were studied. The activated carbon was incorporated at 1%, 5% and 10% w/v, with contact times ranging from 15 minutes to 12 hours. The carbon was subsequently removed by either filtration or centrifugation. By density gradient centrifugation, the adsorption of the normal plant components was determined to occur relatively rapidly (15-30 mins). When a preformed activated carbon filter bed of a certain thickness was employed to remove the suspended carbon, all the TMV and plant components were retained in the bed, while by employing an equivalent quantity of activated carbon and removing by centrifugation, the plant components were retained by the pellet carbon, with the majority of the virus. recovered in the supernatant. The TMV losses by using filtration were notably greater than the losses incurred for either of the icosahedral viruses. Similar to previous studies, Galvez noted that while charcoal was effective at removing the majority of the brown pigments, it had little effect on the green pigments.

### 3. SUMMARY OF THE INVENTION

The present invention relates to the purification of viruses, recombinant virions and virus-like particles from biological source material. Of particular interest are those expressed in plants, preferably through the use of tobamovirus vectors, that may display peptide epitopes on their surface. More specifically it relates to the use of activated carbon and/or high pH and/or high salt concentrations during the purification procedure, as a means to remove protease and nuclease activities that may be detriemtnal to the recombinant virus integrity or its subsequent use.

Other protein adsorbents may be used provided that they have pores sufficiently small so as to exclude viruses and virus-like particles and not adsorb them.

### 4. BRIEF DESCRIPTION OF THE DRAWINGS

FIG. 1. Genomic organization and gene expression strategy of tobamoviruses. Tobamoviruses have a genomic RNA of approximately 6.4 kb. The genomic RNA is used as an mRNA and translated to produce the replicase protein. TMV produces two replicase proteins, with the larger protein being produced by translational readthrough of an amber (UAG) stop codon. All tobamoviruses produce two smaller coterminal subgenomic RNAs (sgRNA). The coat protein is encoded by the 3′-most sgRNA, and the movement protein by the larger sgRNA. The virion RNA and sgRNAs are capped. Tobamovirus RNAs are not polyadenylated, but contain a tRNA-like structure at the 3′ end.

FIG. 2. General diagram depicting five acceptor vectors (pLSB2268, pLSB2269, pLSB2109, pLSB2110, and pLSB 1806) that were employed in the generation of the DJ5 epitope coat protein fusions. All five vectors share the same base vector (pBIT 2150). The region surrounding the coat protein is expanded to show greater detail. Abbreviations: U1 and U5, coat protein derived from TMV U1 and U5 strains, respectively.

FIG. 3. Generalized design of the oligonucleotide pair employed to clone the DJ5 epitope into the TMV 1 and TMV U5 coat protein stains. Note: negative sign represents the reverse complement of the forward nucleotide.

FIG. 4. Generalized flow diagram for the processing of TMV coat protein fusions. Legend; GJ, Initial plant extract “green juice”; S1, Initial supernatant; P1, Initial pellet; S2, Supernatant derived from pellet P1 with alkaline buffer extraction; P2, Final discarded pellet; PEG1, Virus recovered following PEG precipitation; Cent, Centrifugation.

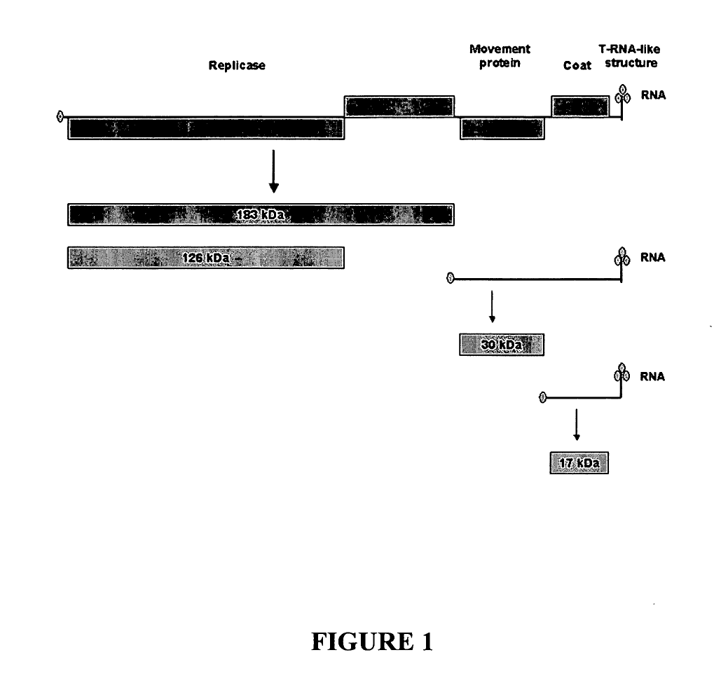

### 5. DETAILED DESCRIPTION OF THE INVENTION

While applicants are not bound by any theory, for simplicity of understanding applicants present proposed mechanisms throught the specification. These mechanisms of action may be incorrect and should not be limiting.

### 6. EXAMPLES

The desgination pLSB#### is used to refer the the DNA plasmid/vector from which the infectious transcript is generated. TMV 26## refers to the recombinant virion, displaying the DJ5 epitope, expressed in planta and purified to generate the vaccine.

### Example 1

### Examples Of Proteolytic Degradation Of Peptide Epitopes Displaced On the Surface Of TMV As Coat Protein Fusions

Degradation can occur to number of peptides fused to the surface of TMV during their extraction and purification. For example, the murine p 15E peptide placed at the N-terminus of the U1 coat protein was purified by pH adjustment as described by Garger et al. (U.S. Pat. Nos. 6,033,895, 6,037,456 and 6,303,779) and Pogue et al. (U.S. Pat. Nos. 6,740,740 and 6,730,306). The mature coat protein fusion amino acid sequence (p 15E DE) was DEKSPWFTTLAG::U1, where U1 represents the wild-type U1 coat protein amino acid sequence, lacking the N-terminal methionine and the N-terminal acidic amino acids (DE) were added to improve recombinant virion expression and solubility. Systemically infected leaf and stalk tissue was macerated in a Waring blender for 1 minute at the high setting with chilled buffer EB1 (0.86 M sodium chloride, containing 0.04% w/v sodium metabisulfite) at a buffer (mL) to tissue (g) ratio of 2:1. The macerated material was strained through four layers of cheesecloth to remove fibrous material. The resultant green juice was adjusted to a pH of 5.0 with phosphoric acid. The pH adjusted green juice was centrifuged at 6,000×G for 3 minutes resulting in two fractions, supernatant S1 and pellet P1. The pellet P1 fraction was resuspended in distilled water using a volume of water equivalent to ½ of the initial green juice volume. The resuspended pellet P1 was adjusted to a pH of 7.5 with sodium hydroxide and centrifuged at 6,000×G for 3 minutes resulting in two fractions, supernatant S2 and pellet P2. Virus was precipitated from both supernatant fractions S1 and S2 by the addition of 4% w/v polyethylene glycol (PEG) 6,000 and 4%. w/v sodium chloride. After incubation at 4° C. (1 hour), precipitated virus was recovered by centrifugation at 10,000×G for 10 minutes. The virus pellet was resuspended in l× PBS, pH 7.4 and clarified by centrifugation at 10,000×G for 3 minutes to yield a final clarified recombinant virion preparation. Aliquots of the green juice, the supernatant S1 and S2 and the final virus preparations, after the clarification spin, were subjected to polyacrylamide gel electrophoresis (PAGE) analysis. The PAGE analysis showed the majority of the principal coat protein band present in the green juice partitioned into the supernatant S2 with low levels present in the supernatant S1. With PEG precipitation of the supernatant S1 and the supernatant S2 and the final clarification spins, virus was further purified from the plant host proteins to yield two substantially pure p15E DE epitope fusion virus preparations. For the S2-derived virion, the major coat protein band co-migrated with the full length coat protein band in the green juice and a minor low molecular weight band, identified by mass spectrometry as a degradation product represented approximately 10-20% of the total protein. In contrast, for the S1-derived virion the observed PAGE profile was reversed; the lower molecular weight truncation product predominated and the full length p15E DE coat protein fusion was the minor product.

Degradation was also observed in the case of a human papillomavirus L2 protein epitope (type 6 or type 11). placed internal to the U1 coat protein C-terminal four amino acids (the GPAT position). The mature coat protein fusion amino acid sequence (HPV 6/11 L2) was U1::GLIEESAIINAGAP::GPAT, where U1 represents the wild-type U1 coat protein amino acid sequence (minus the C-terminal GPAT residues). Systemically infected leaf and stalk tissue, expressing the HPV 6/11 L2 coat protein fusion was harvested and the supernatant S1 was processed such that the temperature was maintained below 10° C., following the procedure outline above for the p15E DE coat protein fusion. A portion of the supernatant S1 was removed and processed at room temperature (˜22° C.) in parallel, to evaluate the influence of temperature on epitope integrity. Aliquots of the green juice, the supernatant S1 and the clarified final virus preparations were subjected to PAGE analysis. The PAGE analysis showed that a single full length coat protein species was present in the green juice and supernatant S1 and when the temperature was maintained below 10° C. throughout processing, the full length HPV 6/11 L2 coat protein fusion constituted >90% of the final virus preparation. However, when the supernatant S1 was adjusted to room temperature degradation of the HPV 6/11 L2 coat protein fusion occurred, such that approximately 50% of the final virus preparation was truncated.

The extent of proteolytic degradation of TMV coat protein fusions that occurred in green juice and supernatant S1 samples was determined to be pH sensitive. This was demonstrated with a human papillomavirus L1 protein epitope (type 16) placed internal to the U1 coat protein C-terminal four amino acids (the GPAT position). The mature coat protein fusion amino acid sequence (HPV 16 L1 I23) was U1::GQPLGVGISGHPLLNKLDDTE::GPAT, where U1 represents the wild-type U1 coat protein amino acid sequence (minus the C-terminal GPAT residues). Systemically infected leaf and stalk tissue, expressing the HPV 16 L1 I23 coat protein fusion was harvested and was processed through to the supernatant S1, with samples of the green juice prior to pH 5.0 adjustment (pH˜5.6) and after adjustment to pH 5.0 taken. The supernatant S1 was divided into three portions; one was retained at pH 5.0, and the other two were adjusted to pH 7.5 and pH 9.0. Aliquots of all samples were taken and heated to 95° C. for 5 minutes in the presence of SDS-PAGE loading dye. The remainder of the samples were placed at 4° C. and stored for 5 days after which a second set of aliquots were taken and heated to 95° C. for 5 minutes in the presence of SDS-PAGE loading dye. All the samples were analyzed by PAGE, to evaluate the stability of the HPV 16 L1 I23 coat protein fusion. The data is summarized in Table 1. The data clearly demonstrates that the protease activity present is maximal under acidic conditions (pH 5), with no full-length species present in either the green juice or the supernatant S1 after storage for 5 days at 4° C. After 5 days at pH 5.6, approximately 30% of the coat protein in the green juice still retained the full HPV 16 L1 I23 epitope, while for the supernatant S1, increasing the pH to 7.5 or above rendered the epitope stable. This result explains the differential stability observed for the p15 E DE epitope coat protein fusion, where degradation occurred for the recombinant virus isolated from the supernatant S1, while the recombinant virus isolated from the supernatant S2 was predominantly full-length. The supernatant S1 is processed at pH 5.0, the pH of maximal proteolytic activity while the supernatant S2 was at pH 7.5, a pH at which protease activity is greatly reduced.

TABLE 1
PAGE analysis of green juice and supernatant S1 samples containing
the HPV 16 L1 I23 coat protein fusion to evaluate the influence of pH
on proteolysis. Boiled samples were taken shortly following extraction
and after 5 days storage at 4° C.

Approximate % of full length
HPV 16 L1 I23 coat protein fusion

| Sample | Day 0 | Day 5 |
|---|---|---|
| Green Juice pH 5.6 | >95% | ˜30% |
| Green Juice pH 5.0 | >95% | <5% |
| Supernatant S1 pH 5.0 | >95% | <5% |
| Supernatant S1 pH 7.5 | >95% | >95% |
| Supernatant S1 pH 9.0 | >95% | >95% |

Epitope degradation has also been observed for purified coat protein fusions stored under pH neutral conditions. This was demonstrated with an epitope from interleukin-1 beta placed internal to the U1 coat protein C-terminal four amino acids (the GPAT position). The mature coat protein fusion amino acid sequence (IL1 □) was U1::AMVQGEESNDKA::GPAT, where U1 represents the wild-type U1 coat protein amino acid sequence (minus the C-terminal GPAT residues). The IL1 □ coat protein fusion was purified to greater that 95% purity from the supernatant S1 following the procedure outlined for the p15E DE epitope coat protein fusion. The virus pellet obtained following PEG precipitation in the presence of sodium chloride was resuspended in 10 mM sodium potassium phosphate, pH 7.2 containing 0.68 M sodium chloride. Two aliquots were prepared and one was stored at −20° C. and the other at 4° C. After 10 days, samples were analyzed by PAGE. For the −20° C. aliquot, a single coat protein band was present of the expected molecular weight. However, in the case of the sample stored at 4° C., <5% of the full length IL1 □ coat protein fusion was present. The proteolytic degradation, which occurred at 4° C. under neutral pH conditions, was confirmed by mass spectrometry. Epitope degradation with storage at −20° C. has also been observed for certain coat protein fusions. Table 2 summarizes stability data for four different TMV coat protein fusions resuspended in phosphate buffered saline, pH 7.4. After 6 months of storage, two of the epitope coat protein fusions, CRPV L2.1 and ROPVL2.2 showed good stability, whereas degradation was evident for the remaining coat protein fusions analyzed, namely HPV 6/11 L2 and ROPVL2.1.

TABLE 2
Analysis of coat protein fusion stability with storage for six months
at −20° C. in phosphate buffered saline, pH 7.4. All the
epitopes were placed internal to the U1 coat protein C-terminal
four amino acids (the GPAT position).

| | | % full length coat | |
|---|---|---|---|
| Epitope | | protein species | |
| Name | Amino acid sequence | Day 0 | 6 months |

| | | | |
|---|---|---|---|
| CRPV L2.1 | GVGPLDIVPEVADPGGPTL | 97.3% | 96.7% |
| HPV 6/11 L2 | GLIEESAIINAGAP | 78.5% | 61.9% |
| ROPV L2.2 | GPAGSSIVPLEEYPAEIPT | 92% | 91% |
| ROPV L2.1 | GVGPLEVIPEAVDPAGSSI | 98% | 73.1% |

The amino acid sequence is for the epitope inserted.
CRPV L2.1, Cottontail rabbit papillomavirus L2 protein epitope 1;
ROPV L2.1, Rabbit oral papillomavirus L2 protein epitope 1;
ROPV L2.2, Rabbit oral papillomavirus L2 protein epitope 2.
The percentage of full length coat protein fusion was determined densitometrically from the Coomassie stained protein gel.

To summarize, the stabilty of a TMV coat protein fusion is epitope dependent. During extraction and processing the stability of the peptide epitope fused to TMV is dependent on pH, with a neutral or high pH (pH 7.0 and above) inhibiting the endogenous protease activity present in the extract in certain cases. For sensitive epitopes, proteolysis is maximal under acidic conditions (pH 5.0). The protease activity is not removed fully by the pH treatment and PEG precipitation procedure outlined. Even in cases where no distinct host protein bands are detected in the final recombinant virus preparation, and storage is under pH neutral conditions, degradation can occur. Furthermore depending on the epitope displayed, the proteolysis can proceed at 4° C. as well as at −20° C.

### Example 2

### Removal Of Plant Host Proteins from TMV Coat Protein Fusion Preparations By Their Selective Adsorption Using Activated Carbon

Experiments were performed with the TMV 150 coat protein fusion, which displayed an epitope from the VP2 protein of canine parvovirus as an N-terminal fusion to the U1 coat protein (Pogue et al. U.S. Pat. Nos. 6,740,740 or 6,730,306). Systemically infected tissue expressing the TMV 150 coat protein fusion was processed to an S1 supernatant at pH 5.0 by one of four procedures:

(1) As described in Example 1 for the p15E DE coat protein fusion, extracting in 0.86 M sodium chloride, containing 0.04% w/v sodium metabisulfite.

(2) As for (1), with the inclusion of a heat treatment step. Briefly, following the adjustment to pH 5+/−0.5, the green juice was heated to 47° C. and held at this temperature for 5 minutes and then cooled to 15° C. The heat-treated green juice was then centrifuged at 6,000×G for 3 minutes.

(3) As for (1), but extracting in deionized water containing 0.04% w/v sodium metabisulfite.

(4) As for (3), with the inclusion of a heat treatment step. Briefly, following the adjustment to pH 5+/−0.5, the green juice was heated to 47° C. and held at this temperature for 5 minutes and then cooled to 15° C. The heat-treated green juice was then centrifuged at 6,000×G for 3 minutes.

The S1 supernatants from each of these treatments was combined with activated carbon at 5% w/v and mixed for 1 hour at 4° C. The activated carbon was removed by centrifugation at 2000×g for 30 minutes. In control samples the activated carbon was omitted. The supernatants were analyzed by PAGE and the results are summarized in Table 3.

TABLE 3
Influence of sodium chloride during extraction and heat
treatment on the effectiveness of activated carbon (AC)
as a method of purifying a TMV coat protein fusion.

| | | | Activated | TMV 150 recovery |
|---|---|---|---|---|
| Treatment | Sodium | Heat | carbon | in treated |
| condition | chloride | Treatment | treatment | supernatant S1 |
| (1) | YES | NO | Control | 98% |
| | | | 5% w/v AC | 83% |
| (2) | YES | YES | Control | 95% |
| | | | 5% w/v AC | 80% |
| (3) | NO | NO | Control | 0% |
| | | | 5% w/v AC | 61% |
| (4) | NO | YES | Control | 0% |
| | | | 5% w/v AC | 0% |

By PAGE analysis, the activated carbon treatment effectively removed all the plant host proteins from the supernatant S1 that were otherwise present in the control samples. The level of TMV 150 coat protein fusion remaining in the supernatant varied from 0% to over 80% depending on the procedure. With the extended centrifugation time employed to ensure complete removal of the activated carbon, complete precipitation of the TMV 150 coat protein fusion occurred in the samples extracted in the absence of sodium chloride; condition (4). This precipitation was the result of virus aggregation at pH 5.0, which is close to the isoelectric point for the TMV 150 fusion. Of note was the fact that for the extraction in deionized water, where no heat treatment was performed, activated carbon treatment resulted in the recovery of 61% of the TMV 150 fusion. This suggests that in the absence of heat treatment, activated carbon treatment adsorbs plant component(s) that influence the aggregation of TMV 150 under acidic pH conditions. When salt was included during the extraction procedure (conditions (1) and (2)), TMV 150 solubility was improved considerably, with essentially all the virion remaining in the supernatant following the 30 minute centrifugation. The presence of salt presumably disrupts the inter-virion associations that result in aggregation. In the presence of sodium chloride, the recovery of the TMV 150 in the final supernatant was not influenced by heat treatment and recoveries were at or above 80%. These results provide proof of principle for TMV purification with the aid of activated carbon.

Some processing parameters e.g. presence/absence of salt and heat treatment, also influence virus recovery. An additional condition to those listed in Table 3 was tested; TMV 150 virus in deionized water and alkaline pH conditions (pH 8.6). Since the sample pH was several units above the virus isoelectric point the precipitation observed in the control sample of conditions (3) and (4) did not occur; ˜98% of the TMV 150 remained in the supernatant. With activated carbon treatment, effective host protein adsorption was achieved and 72% of the starting virus was recovered in the supernatant.

A further experiment was conducted to evaluate the influence of activated carbon source. Systemically infected tissue expressing the TMV 150 coat protein fusion was processed to an S1 supernatant at pH 5.0 by one of one of two procedures:

(1A) As described in Example 1 for the p15E DE coat protein fusion, extracting in 0.86 M sodium chloride, containing 0.04% w/v sodium metabisulfite.

(2A) As for (1A), with the inclusion of a heat treatment step. Briefly, following the adjustment to pH 5+/−0.5, the green juice was heated to 47° C. and held at this temperature for 5 minutes and then cooled to 15° C. The heat-treated green juice was then centrifuged at 6,000×G for 3 minutes.

Three different grades of activated carbon were tested, Norit S51 FF, Nuchar SA-20 and Sigma C-5620. The activated carbon was employed at 5% w/v and contacted with the S1 supernatants for 1 hour at 4° C., and removed by centrifugation at 2,500×g for 30 minutes or alternatively by filtration through a glass fiber filter. Table 4 summarizes the results. Overall host protein removal by the activated carbon was comparable across all conditions. Losses of TMV 150 with activated carbon removal by filtration were substantial, indicating entrapment of the virus rods in the activated carbon bed. The consistently higher losses with heat treatment suggests that this processing step results in a higher degree of virion aggregation. TMV 150 recoveries with activated carbon removal by centrifugation were notably higher than with filtration and differences in performance were evident between the grades of activated carbon tested. In particular the Nuchar SA-20 grade of activated carbon resulted in consistently lower recoveries relative to the Norit S51 FF and Sigma C-5620 grades. The negative influence of heat treatment on TMV 150 recovery was also seen in the samples where centrifugation was used for activated carbon removal.

TABLE 4
Influence of heat treatment, activated carbon grade and activated carbon removal
method (centrifugation vs. filtration) on the recovery of TMV 150 coat protein fusion.
Activated carbon grades employed were Norit S51 FF, Nuchar SA-20 and Sigma C-5620.

| | | | TMV 150 recovery in treated supernatant S1 | | | | | |
|---|---|---|---|---|---|---|---|---|
| Treatment | Sodium | Heat | Centrifugation | | | Filtration | | |
| condition | chloride | Treatment | S51 FF | C-5620 | SA-20 | S51 FF | C-5620 | SA-20 |
| (1A) | YES | NO | 77% | 77% | 66% | 31% | 27% | 12% |
| (2A) | YES | YES | 51% | 51% | 23% | 7% | 11% | 0% |

The studies with activation carbon based purification of the TMV 150 coat protein fusion provide proof of principal for the selective adsorption of globular plant host proteins by activated carbon, resulting in a purified virus preparation. Since the host proteases responsible for the proteolytic degradation described in Example 1 are globular proteins, activated carbon treatment may serve are a means of protease removal, thereby improving TMV coat protein fusion stability during processing and/or storage.

### Example 3

### Activated Carbon As a Means for Preventing Proteolytic Degradation Of TMV Coat Protein Fusions During Processing

For the HPV 16 L1 I23 TMV U1 coat protein fusion, proteolytic degradation was shown to occur under acidic conditions during processing (Example 1). To evaluate the ability of activated carbon to adsorb the proteases present in the supernatant S1, as hypothesized in Example 2, the following experiment was performed. Systemically infected tissue expressing the HPV 16 L1 I23 TMV U1 coat protein fusion was processed to an S1 supernatant at pH 5.0 following the procedure outlined in Example 1 for the p15 E DE coat protein fusion. This S1 supernatant was divided in two and one half was processed according to Example 1 to yield a PEG purified recombinant virus preparation that was resuspended in pH 7 buffer and stored at 4° C. (S1 PEG 1 pH 7). Activated carbon at 5% w/v was added to the other half of the pH 5 S1 supernatant and following mixing for 1 hour at 4° C., was removed by centrifugation. The activated carbon treated S1 was maintained at pH 5 and was stored at 4° C. (S1 AC pH 5). For both the S1 PEG 1 pH 7 and S1 AC pH 5 material, samples were taken after 1,2,7 and 11 days and boiled at 95° C. for 5 minutes in SDS-PAGE loading dye. PAGE analysis was performed on the samples. For the S1 AC pH 5 sample, the HPV 16 L1 I23 TMV U1 coat protein fusion integrity was maintained for the 11 days testing period and no increase in the truncation species (˜5-10% of initial sample) was observed. In contrast, for the S1 PEG 1 pH 7 sample increased degradation was evident after storage for 24 hours at 4° C. and by day 11, the truncated species constituted over 60% of the coat protein present. By demonstrating that activated carbon treatment prevented the proteolytic degradation of the HPV 16 L1 I23 TMV U1 coat protein fusion when stored under conditions of optimal protease activity, this experiment provides proof of principle for activated carbon treatment as a means to improve recombinant virus stability.

### Example 4

### Activated Carbon As a Means for Preventing Proteolytic Degradation Of Purified TMV Coat Protein Fusions

Low levels of protease(s), 0.5% or less, may be present in the final virus preparations obtained by the purification procedure outlined in Example 1 for the p15E DE TMV coat protein fusion. The presence of these proteases was demonstrated in Example 1 by the reduction in the percentage of full length coat protein fusions with storage. To evaluate activated carbon treatment as a method to remove proteases associated with final purified virus preparations the following experiment was performed. The purified PEG precipitated virus (purity >95%, resuspended in a pH 7 buffer) displaying the HPV 6/11 L2 epitope was divided into three equal portions and adjusted to 1% w/v activated carbon, 5% w/v activated carbon or left untreated. After mixing for 1 hour at 4° C., the activated carbon was removed by centrifugation and each supernatant, together with the control material was further subdivided in two, giving a total of six samples. One of each treatment sample was adjusted from pH 7 to pH 5 by the addition of phosphoric acid and the samples were stored at room temperature. Aliquots were taken after 24 hours and 8 days, boiled at 95° C. for 5 minutes in SDS-PAGE loading dye and analyzed by PAGE. The data is summarized in Table 5. No degradation was observed in any of the samples stored at pH 7, whereas there were notable differences for the pH 5 samples. Specifically for the control, only 30-40% of the full length species remained. Treatment with 1% w/v activated carbon improved coat protein fusion stability considerably with only 5-10% truncation, while 5% w/v activated carbon treatment afforded complete protection. The overall recoveries for the activated carbon treatments were 60% and 80% for the 5% w/v and 1% w/v activated carbon treatments respectively. This example demonstrates that activated carbon treatment can successfully remove low levels of protease activity from purified virus preparations to improve stability with storage.

TABLE 5
Effect of pH and activated carbon treatment on the stability of
purified HPV 6/11 L2 epitope coat protein fusion with storage
at room temperature. For each condition the approximate
percentage of intact fusion after 24 hours and 8 days of
storage at room temperature is indicated.

| | |
|---|---|
| | % full length coat |
| Sample treatment | protein species |

| Activated carbon | Storage pH | 24 hours | 8 days |
|---|---|---|---|
| 0% w/v (control) | pH 5 | >90% | 30-40% |
| | pH 7 | >95% | >95% |
| 1% w/v | pH 5 | >95% | 80-90% |
| | pH 7 | >95% | >95% |
| 5% w/v | pH 5 | >95% | >95% |
| | pH 7 | >95% | >95% |

### Example 5

### Activated Carbon As a Means for Removing Ribonuclease Activity Associated with Purified TMV and TMV Coat Protein Fusions

The procedures outlined in Example 3 and 4EA can be extended to nuclease removal from purified TMV and TMV coat protein fusion preparations. Ribonuclease and deoxyribonuclease removal is of importance when coat protein is generated from the virus preparation, for use in transcript encapsidations. If the coat protein preparation is contaminated with nuclease activity, it will result in transcript degradation. Activated carbon, employed at 1% w/v and 5% w/v has been shown to effectively remove all associated ribonuclease activity from wild-type TMV and the TMV coat protein fusions listed in Table 6. Table 6 also indicates that the observed virus recoveries were at 80% or above.

TABLE 6
Recovery of wild-type TMV and a number of TMV coat protein
fusions following activated carbon treatment to remove
associated ribonuclease activity. The ELDKWAS epitope was
derived from Human Immunodeficiency virus (HIV), other epitopes
were from HPV, human papillomavirus, IL1B, Interleukin
1 beta(IL1 □). The integrin binding motif was derived
from the adenovirus capsid protein.

| Fusion | Amino acid sequence | Mg processed | Recovery |
|---|---|---|---|
| ELDKWAS | ELDKWAS | 130 mg | 83% |
| HPV 6/11 | GLIEESAIINAGAP | 120 mg | 86% |
| Integrin binding | RGD | 330 mg | 80% |
| IL1 β | AMVQGEESNDKA | 160 mg | 80% |
| Wild-type U1 | N/A | 760 mg | 83% |

A more detailed description of the use of activated carbon in the removal of ribonuclease associated with wild-type TMV and TMV coat protein fusions follows. The original methodology for the generation of viral coat protein for RNA encapsidation was detailed by Fraenkel-Conrat (Fraenkel-Conrat, H. (1957). Degradation of tobacco mosaic virus with acetic acid. Virology 4, 1-4.) using tobacco mosaic virus. The procedure involves the following steps:
1. Combine the purified TMV, at a concentration of 10-50 mg/ml with glacial acetic acid, to obtain a final acetic acid concentration of 67% v/v. Incubate on ice for 15 to 60 minutes. The acetic acid treatment disassociates the virus and causes the genomic RNA to aggregate and precipitate.
2. Centrifuge to pellet the precipitated RNA, resulting in a water-clear free coat protein solution
3. Dialyze the coat protein preparation extensively against water to remove the acetic acid (2-3 days). With the removal of the acetic acid, the pH of the free coat protein increases, until the p1 of the coat protein (˜pH 4.5 to pH 4.7) is reached. At this point the coat protein aggregates and precipitates. To ensure complete coat protein precipitation, a few drops of 3 M sodium acetate, pH 4.7 can be added.
4. Ultracentrifugation is employed to recover the coat protein pellet, which is subsequently resuspended in a reduced volume to obtain the desired concentration (typically 5-10 mg/ml).
5. To resolubilize the coat protein, sodium hydroxide is added until the pH of the solution is between 7 and 8.
6. A second ultracentrifugation is then performed to remove undegraded virus.

This procedure was effective at obtaining coat protein preparations that were capable of reassembling onto isolated TMV genomic RNA (Fraenkel-Conrat, H., and Singer, B. (1959). Reconstitution of tobacco mosaic virus III. improved methods and the use of mixed nucleic acids. Biochim Biophys Acta 33, 359-370). However, Fraenkel-Conrat and Singer noted that ribonuclease contamination could be an issue, which reduced the efficiency of the virus reassembly, i.e. the viral RNA encapsidation, due to degradation of the RNA scaffold. Fraenkel-Conrat and Singer indicated that by replacing the 0.1 M phosphate encapsidation buffer with 0.1 M pyrophosphate, the ribonuclease activity was reduced. The final free TMV coat protein preparation is one possible source of ribonuclease contamination, which can reduce the coat protein effectiveness, owing to the degradation of the RNA scaffold to which the coat protein is added.

The RNA scaffold in question can take several forms, provided the nucleotide sequence corresponding to the origin of assembly (OAS) is present. In the TMV genome the OAS is located approximately 900 nucleotides from the 3′ terminus. The coat protein forms a 34 subunit oligomer (termed a 20S disk), which reacts specifically with the OAS. Additional 20S disks are added and packaging (encapsidation) is then completed by rod elongation both in the 5′ and 3′ directions. For TMV-based expression systems where an additional subgenomic promoter and a coding sequence for a foreign protein are introduced, the encapsidation of the transcript confers increased stability to the RNA, protecting it from degradation during handling and inoculation onto host plants. Through genetic engineering, the OAS can be combined with foreign nucleotide sequences, to generate vectors that will express in other hosts, such as mammalian cells. These vectors can potentially be used as therapeutics, to direct the transient expression of a biologically relevant protein. Furthermore, the coat protein itself can be modified to display foreign epitopes on its surface. The foreign peptide may have an immunological function or modify the coat protein to permit the attachment of peptides or whole foreign proteins to the surface of the reassembled capsids. Once application of this is the creation of multifunctional vaccines, having both a protein component, displayed on the capsid surface and a functional nucleic acid component, permitting transient expression of an additional protein or proteins in cells which uptake the reassembled product. In all the compositions described above, it is critical that the coat protein preparation employed be ribonuclease free, to ensure that the integrity of the RNA being encapsidated is maintained.

In developing robust methods for removing ribonuclease activity from coat protein preparations, the following criteria were established. The procedure was required to function independent of the epitope displayed on the coat protein surface and to generate a reassembly competent coat protein preparation. In addition the procedure should not negatively impact coat protein recovery. To evaluate the level of ribonuclease present in a given sample a qualitative agarose gel electrophoresis assay was used. The sample of interest, e.g. starting virus, in process sample, final free coat protein, water or buffer, was incubated with purified TMV genomic RNA (isolated from TMV using the RNeasy kit (Qiagen, Valencia, Calif.)) for 2-4 hours at room temperature, with a final TMV RNA concentration of 120 mg/ml in each sample. An aliquot of each sample/RNA mixture was combined with one half volume of RLT buffer (Qiagen), to inactivate any ribonuclease present and prevent further degradation, and three volumes of Gel loading buffer (Ambion, Austin, Tex.). A volume of 10 ul was analyzed on a 1.2% agarose TBE gel, and the nucleic acid visualized by ethidium bromide staining. The 6400 nucleotide TMV genomic RNA migrates as a single well defined band with an apparent molecular weight of 3 kb relative to a 1 kb DNA ladder (NEB, Ipswich, Mass.). When ribonucleases are present degradation of the RNA occurs and the nucleic acids migrates as a diffuse band. The molecular weight depends on the extent of degradation and in cases of severe ribonuclease contamination, no RNA band is detectable.

Initially, treatment of the starting TMV preparation with diethylpyrocarbonate (DEPC) was tested. DEPC is commonly used to inactivate ribonucleases in water and buffers. It functions by derivitizing histidines & tyrosines in the active site of ribonucleases. However, these modifications will not be limited to ribonucleases. The N and C terminus of the TMV capsid are surface exposed and two tyrosines, at position 3 and 140 are potential sites for modification. In addition, if epitopes fused to the TMV capsid protein contain either histidines or tyrosines, these could also be potentially modified.

Preliminary testing of DEPC as a means for ribonuclease removal involved coat protein preparations derived from wild-type TMV U1, as well as three TMV U1 coat protein fusions. For one of the constructs, the amino acid sequence ELDKWAS, derived from the gp41 protein of HIV was fused to the N-terminus of the U1 coat protein. For the other two constructs, the Myc epitope was displayed at either the N of C terminus of the U1 coat protein. The coat protein preparations were incubated with either 0.1% or 0.5% v/v of DEPC at room temperature for 16 hours and the samples then dialyzed against 0.1 M Tris, to inactivate residual DEPC. Aliquots of the untreated and treated coat protein preparations were then combined with TMV genomic RNA, and following a 4 hour incubation, the integrity of the added RNA was assessed by agarose gel electrophoresis as outlined above. Ribonucleases were present in all samples, however, for the samples incubated with DEPC, RNA degradation was retarded. A series of optimization studies were performed and the following procedure was defined for treatment of virus samples contaminated with ribonuclease:
1. The virus was diluted to 1 mg/ml and dialyzed into potassium phosphate pH 6. A pH of 6 was chosen as DEPC modification is reported to be specific for histidine at this pH, thereby improving the specificity of the carbethoxylation reaction. At other pHs, DEPC is reported to react with methionine, lysine, serine and tyrosine.
2. DEPC was added to 0.5% v/v and the sample incubated for 16 hours at 37° C.
3. The sample was dialyzed against Tris, pH 7, to neutralize residual DEPC and subsequently concentrated by PEG precipitation.

These conditions were confirmed to effectively inactivate ribonuclease by TMV RNA addition/agarose gel electrophoresis, when tested with several TMV U1 virus preparations and the coat protein fusions listed in Table 7. When the virus preparations were analyzed by mass spectrometry, peaks were observed in the spectra corresponding to +73 Da and +146 Da additions. These additions correspond to carbethoxylation by DEPC and the modifications are most likely occurring at the surface exposed tyrosine residues. The processing conditions were therefore not specific for histidine modification. Since modification to the coat protein was occurring, it was necessary to determine if these modifications altered the ability of the coat protein generated from the DEPC-treated virus to reassemble. The virus samples listed in Table 7 were disassociated by acetic acid treatment as outlined above and the coat protein isolated. The UV adsorption spectrum of the coat protein was obtained and a ratio of the absorbance (optical density; OD) at 250 nm and 280 nm taken. A ratio above 2 is expected for free coat protein and was obtained for the three coat protein preparations derived from DEPC-treated virus (Table 7).

TABLE 7
TMV samples for which DEPC-treatment effectively inactivated
contaminating ribonuclease activity and modifications to the coat
protein as a result of the treatment, as determined by mass
spectrometry. Coat protein was prepared from a subset of the
treated and untreated virus preparations. For UV adsorption
spectrum for these coat protein preparations was
obtained and the OD 250/280 ratio determined.

| | | | | | OD |
|---|---|---|---|---|---|
| | | | | | 250/ |
| | | | Principal | | 280 |
| Fusion | DEPC | Expected MW | Peak | Other peaks | ratio |
| WT | NO | 17533 | 17536 | — | >2.0 |
| U1 | | −Met + acetyl | Match | | |
| | YES | Ser | 17535 | 17607 | 2.33 |
| | | | Match | +73 Da | |
| IL1B GPAT | NO | 18795 | 18797 | 18734 | 2.36 |
| | | −Met + acetyl | Match | No Match | |
| | YES | Ser | 18794 | 18863/18939 | 2.26 |
| | | | Match | +73/+146 Da | |
| Integrin | NO | 18425 | 18434 | — | 2.30 |
| GPAT | | −Met + acetyl | Match | | |
| | YES | Ser | 18435 | 18506 | 2.60 |
| | | | Match | +73 Da | |

Next the DEPC-modified coat protein preparations were evaluated in encapsidation reactions, using TMV genomic RNA as a scaffold, to determine if the coat protein modifications detected by mass spectrometry interfered with the coat protein's ability to reassemble. The reassembly reactions were monitored by the following metrics:
(1) Change in absorbance at 310 nm following coat protein addition to the RNA. With RNA encapsidation by coat protein and rod formation, the absorbance at 310 nm increases, with the absorbance being proportional to the average rod length.
(2) The local lesion host assay on N. tabacum cv. Xanthi (N). Encapsidation of genomic RNA results in a significant increase in infectivity which is measured as an increase in the lesion numbers detected.
(3) Electron microscopy to visualize the reassembled rods directly.

When the coat protein derived from the untreated virus (ribonuclease removed by activated carbon) was compared to that from DEPC-treated virus, no difference was observed for the wild-type (WT) U1 or the integrin coat protein fusion: An increase in absorbance at 310 nm was observed following coat protein addition to the RNA and when analyzed by electron microscopy, full length (300 nm) rods were detectable. In addition, encapsidation with the DEPC-modified coat protein resulted in a notable increase in lesion numbers relative to the TMV genomic RNA alone (Table 8), similar to that observed with the untreated (control) coat protein. If the carbethoxylation of the reassembled product is unacceptable, the N-carboxyl group can be removed by treatment with hydroxylamine. However, it should be noted that this treatment recovers the original amino acid in the cases of histidine and tyrosine, but not of other potentially modified residues.

For the third coat protein tested, which displayed an epitope from IL1β, no reassembled rods were detected by electron microscopy when the DEPC-treated preparation was employed. Furthermore, the absorbance at 310 nm did not increase with time, after coat protein addition to the RNA and when inoculated onto N. tabacum cv. Xanthi, lesions numbers obtained were comparable to RNA alone (Table 8). In contrast, efficient reassembly was obtained for the IL1β, coat protein that was not subject to DEPC treatment (Table 8). In summary, while DEPC is effective at removing ribonuclease activity, the procedure was not broadly applicable, as for certain coat protein fusions, the DEPC modifications appeared to interfere with coat protein reassembly.

TABLE 8
Evaluation of TMV genomic RNA encapsidation reactions
employing coat protein (CP) derived from DEPC-treated virus
and from control coat protein preparations (no DEPC treatment).

| | | DEPC-treated |
|---|---|---|
| | Control CP | CP |
| Virus | >300 | |
| RNA | 5 +/− 3 | |
| RNA + WT U1 | >300 | >300 |
| CP | | |
| RNA + Integrin | >300 | >300 |
| CP | | |
| RNA + IL1B CP | >300 | 2 +/− 2 |

In parallel, activated carbon was evaluated as a method to remove ribonuclease activity from virus preparations, prior to acetic acid treatment to generate coat protein. Similar to the removal of proteases and other soluble host-derived protein, preliminary experiments indicated that activated carbon was capable at adsorbing ribonucleases (RNases), resulting in RNase free virus preparations, as assessed by the TMV RNA addition/agarose gel electrophoresis method. Next, experiments were conducted to optimize virus recovery. Virus displaying the ELDKWAS epitope was diluted to 1 mg/ml and treated with activated carbon (Norit KB-FF or Nuchar SA-20) at 1% w/v to 5% w/v. Following a 1 hour incubation at 4° C., the activated carbon was removed by centrifugation and the protein concentration of the recovered supernatant determined. The volume recovered was also measured to determine recovery. The results are summarized in Table 9.

TABLE 9
Virus recovery following activated carbon treatment of a TMV coat
protein fusion displaying the ELDKWAS epitope. Two different
activated carbon grades were tested, Norit KB-FF and Nuchar SA-20.

| % w/v activated carbon/Grade | % Recovery | Ribonuclease activity |
|---|---|---|
| 0% Norit KB-FF | 100% | +++ |
| 1% Norit KB-FF | 85% | −−− |
| 2.5% Norit KB-FF | 70% | −−− |
| 3.5% Norit KB-FF | 58% | −−− |
| 5% Norit KB-FF | 40% | −−− |
| 0% Nuchar SA-20 | 100% | +++ |
| 1% Nuchar SA-20 | 87% | −−− |
| 2.5% Nuchar SA-20 | 65% | −−− |
| 3.5% Nuchar SA-20 | 32% | −−− |
| 5% Nuchar SA-20 | 10% | −−− |

The initial virus and virus treated with the different levels of activated were tested for ribonuclease activity;
+++, ribonuclease detected;
−−−, no ribonuclease detected.

At the lower concentration of activated carbon tested, recoveries with both grades of activated were comparable and by the TMV RNA addition/agarose gel electrophoresis method, ribonuclease activity was effectively removed at the lowest quantity of activated carbon employed (1% w/v). At higher activated carbon concentrations, the extent of virus loss was dependent on the grade of activated carbon employed. Since no benefit was observed at the higher levels of activated carbon, 1% w/v activated carbon was tested with a number of different virus preparations (at 1 mg/ml), some of which displayed foreign epitopes (Table 6). In all cases, recoveries comparable to those obtained in Table 9 were observed and when the activated carbon treated coat protein was tested, all ribonuclease activity initially present was removed. As expected, when analyzed by mass spectrometry, no modifications to the coat protein were observed, a distinct advantage over the DEPC treatment method, as the procedure is therefore more broadly applicable.

The activated carbon treated wild-type U1 virus was processed by treatment with 67% acetic acid, to obtain free coat protein that was tested with a number of different RNA scaffolds. The lesion numbers obtained following encapsidation were compared to inoculation with the RNA transcript alone, or to transcript encapsidated with coat protein preparation prepared without prior activated carbon treatment, which contained detectable levels of ribonuclease activity. One transcript employed in the tests was a TMV expression vector which expressed green fluorescent protein (GFP). When this transcript was employed GFP spots, denoting infection sites, could be scored under ultraviolet illumination. This permitted encapsidated transcript evaluation on N. benthamiana, a production host for recombinant proteins expressed using TMV-expression vectors. The data from multiple experiments is summarized in Table 10.

TABLE 10
Evaluation of TMV U1 coat protein (CP) processed from virus
treated with activated carbon (A/C) to remove ribonuclease or
from untreated virus (no A/C). RNA alone or encapsidated
RNA were inoculated onto either N. tabacum cv. Xanthi
(N) (local lesion assay) or N benthamiana, with inoculations
normalized to the same volume of RNA transcript. Five to
six days post inoculation local lesion or GFP spots were
scored and the averages from at least 6 independent
inoculations are reported. The TMV vectors tested expressed either
GFP (green fluorescent protein) of LAL (lysosomal acid lipase).

| | | Average number of | |
|---|---|---|---|
| | | lesions/GFP spots | |
| Inoculum | Transcript | Local lesion assay | N benthamiana |

Experiment #1

| | | | |
|---|---|---|---|
| RNA | GFP TMV vector | 36 | 115 |
| RNA + CP | GFP TMV vector | 9 | 22 |
| (no A/C) | | | |

Experiment #2

| | | | |
|---|---|---|---|
| RNA | GFP TMV vector | 45 | 41 |
| RNA + CP (A/C) | GFP TMV vector | 204 | 176 |

Experiment #3

| | | | |
|---|---|---|---|
| RNA | LAL TMV vector | ˜50 | Not tested |
| RNA + CP | LAL TMV vector | 0.3 | Not tested |
| (no A/C) | | | |
| RNA + CP (A/C) | LAL TMV vector | 172 | Not tested |

When coat protein was derived from virus without prior ribonuclease removal, a 4-6 fold drop in average lesion /GFP spot number was observed when this coat protein was employed for encapsidation, relative to RNA alone (Experiment #1, Table 10). In the case of the LAL transcript, the loss in infectivity was even more notable, indicating that this particular transcript was especially sensitive to ribonuclease (Experiment #3, Table 10). In contrast, when the coat protein employed was derived from activated-carbon treated virus and was therefore ribonuclease free, a 3 to 5 fold increase in infectivity was observed on both plant hosts tested, for both the GFP and the LAL transcripts. In summary, these results demonstrate the effectiveness of activated carbon treatment for ribonuclease removal and show that encapsidation with ribonuclease-free coat protein results in an improved inoculum, relative to RNA alone.

### Example 6

### Activated Carbon As a Means For Purifying Icosahedral Viruses and Virus-like Particles: Hepatitis C Core Antigen

To evaluate the usefulness of activated carbon in the purification of icosahedral viruses and virus-like particles, tissue homogenates from plants infected with a tobacco mosaic virus-derived vector, expressing core antigen particles of hepatitis B (HBcAg), were processed as outlined below. HBcAg forms the icosahedral nucleocapsid of the hepatitis B virus and the particles have a diameter of 27 nm, with T=3 or T=4 symmetry. With transient expression in N. benthamiana, HBcAg accumulated to 200 □g per gram infected tissue. The tobacco mosaic virus vector expressing HBcAg was designated pLSB2612. For the processing, systemically infected N. benthamiana tissue was combined with 3 volumes of 50 mM acetate buffer, pH 4.8, 400 mM NaCl, containing 0.4% w/v sodium metabisulfite and a Waring blender employed to homogenize the tissue. The extract was passed through cheesecloth and the resulting green juice (GJ) was centrifuged at 10,000×g for 15 minutes. Under acidic conditions, the fraction 1 proteins and associated pigment coagulate, and are removed by the centrifugation, resulting in a clarified extract. A portion of the clarified extract was dialyzed into 50 mM acetate, pH 4.8, to eliminate the sodium chloride and other low molecular weight solutes. Dialysis into an alkaline buffer lacking sodium chloride (20 mM tris(hydroxymethyl)aminomethane, pH 9) was also performed. The dialyzed supernatants were further subdivided and sodium chloride added to 300 mM. The final condition tested was to adjust the clarified extract to pH 9, without dialysis, using sodium hydroxide. A summary of these conditions is provided in Table 11. When a precipitate formed as a result of the pH change or dialysis, it was removed by centrifugation prior to the activated carbon testing. The samples were combined with 5% w/v activated carbon (Sigma, St. Louis, Mo.), and following a contact time of 1 hour at 4° C., the activated carbon was removed by centrifugation at 8,000×g for 5 minutes. The water clear supernatants were transferred to new containers and were analyzed by protein gel electrophoresis on a 10-20% tris glycine gel, with Coomassie blue staining employed to visualize the proteins. Control samples for all buffer conditions, where no activated carbon was added, were processed in parallel

TABLE 11
Summary of buffer conditions tested with activated carbon
treatment for the HBcAg containing clarified extract
obtained from infected N. benthamiana tissue.

| | | | NaCl | Other buffer |
|---|---|---|---|---|
| Condition | Sample | Approximate pH | concentration | components |
| A | Clarified extract | pH 5 | ˜300 mM | 50 mM acetate, |
| | | | | 0.4% w/v sodium |
| | | | | metabisulfite |
| B | Clarified extract | pH 9 | ˜300 mM | 50 mM acetate, |
| | | | | 0.4% w/v sodium |
| | | | | metabisulfite |
| C | Dialyzed clarified | pH 5 | — | 50 mM sodium |
| | extract | | | acetate |
| D | Dialyzed clarified | pH 5 | 300 mM | 50 mM sodium |
| | extract | | | acetate |
| E | Dialyzed clarified | pH 9 | — | 20 mM Tris base |
| | extract | | | |
| F | Dialyzed clarified | pH 9 | 300 mM | 20 mM Tris base |
| | extract | | | |

The gel analysis of the samples indicated that the recovery of wild-type TMV (coat protein migrated at ˜20 kDa) was comparable (>90%) under all the conditions tested. This suggests that for rod shaped capsids such as TMV, the recovery from activated carbon treatment is relatively insensitive to pH (acidic vs. alkaline conditions) and is compatible with sodium chloride, up to a concentration of at least 300 mM. It should be noted, however, that the display of foreign epitopes on the TMV capsid surface can effect TMV recovery as a function of pH, as noted in Table 3. In the case of TMV coat protein fusions, only certain pH and salt combinations may therefore function effectively with activated carbon treatment.

In contrast to wild-type TMV, the recovery of HBcAg (coat protein migrated at ˜22 kDa; no foreign epitopes displayed) following activated carbon treatment was affected by both pH and salt concentration. For the control samples, no loss in HBcAg was observed with centrifugation under any of the buffer conditions listed in Table 11. For the activated carbon-treated salt- containing clarified extracts (Conditions A & B), HBcAg recovery was improved substantially when the sample was adjusted to pH 9. Adsorption of HBcAg by the activated carbon was also observed in the pH 5 dialyzed samples (Conditions C & D), with complete removal when 300 mM NaCl was present. When the clarified extract was dialyzed into a pH 9 buffer (Condition E), 80-90% of the starting HBcAg was recovered following activated carbon treatment. Under alkaline conditions, the overall HBcAg surface charge is negative. This, combined with the macromolecular structure of the HBcAg VLP, results in exclusion of the majority of the particles from the negatively charged pores of the activated carbon. Recovery at pH 9 was reduced by the inclusion of sodium chloride (Conditions F), indicating that an increase in buffer ionic strength promotes HBcAg adsorption to the activated carbon, most likely as a result of the neutralization of ionic repulsions. At pH 5, the net charge of the HBcAg VLP surface is positive, promoting adsorption by activated carbon, which is negatively charged over a broad pH range.

From the Coomassie staining gel, plant proteins over the full molecular weight range (<6 kDA to ˜220 kDa) were effectively removed by activated carbon treatment under all the conditions listed in Table 11. The plant-derived globular proteins can diffuse freely into the pores, and are retained by short-range attractive Van der Waals forces. However, when the gel was subsequently silver stained, to improve the detection of low levels of protein, differentiation between the conditions was observed. Residual levels of plant proteins were detected in the dialyzed pH 9 sample lacking NaCl (Condition E) and these protein were effectively adsorbed when NaCl was included (Condition F). For the dialyzed samples at pH 5 (Conditions C & D), all plant proteins were adsorbed. This demonstrates that plant protein removal was also affected by pH and ionic strength although less so than HBcAg. The level of host protein removal from solution can be adjusted by changing the buffer conductivity: addition of salt improves host protein adsorption to the activated carbon by counteracting ionic repulsions. However, this increased purity must be balanced against higher HBcAg losses. In summary, by optimizing the pH and ionic conductivity of the sample, i.e. by employing alkaline conditions and a NaCl concentration in the 0 to 300 mM range, activated carbon was effective at purifying HBcAg from the plant host proteins.

Subsequently, a series of activated carbon grades were tested to compare recoveries as well as host protein removal. The dialyzed clarified extract (Condition F) was employed and in addition to the Sigma and KB-FF (Norit, Marshall, Tex.) grades of activated carbon previously tested, the following grades were evaluated; Norit S51FF, Norit G60 and Nuchar SA-20, Nuchar SA-1500 and Nuchar RGC (all three from Westvaco, Covington, Va.). The activated carbons were tested at 5% w/v, with a 1 hour contact time at 4° C. Analysis by gel electrophoresis indicated comparable recovery of the HBcAg with all the activated carbon grades. Plant protein adsorption was also effective with the activated carbon grades tested, although from the silver stain of the gels, maximal removal of soluble plant proteins from the supernatant was achieved with the Norit S51FF, Nuchar SA-20 and SA-1500 grades.

To separate the TMV and HBcAg capsids, differential precipitation using polyethylene glycol (PEG) can be performed. Rod-shaped capsids, such as TMV, require a lower PEG concentration for virion precipitation, when compared to icosahedral capsids, such as HBcAg. The precipitation by PEG is facilitated by the presence of NaCl, which reduces the PEG concentration required to obtain capsid aggregation and precipitation. For this reason, Condition F material, after activated carbon treatment, was carried forward and a portion was adjusted to pH 5, to evaluate the influence of pH. Following the addition of PEG to 4% w/v and incubation for 1 hour at 4° C., an initial centrifugation (10,000×g for 10 minutes) was performed. The PEG concentration was then adjusted to 10% w/v, the samples stored for 16 hours at 4° C. and a final centrifugation performed (10,000×g for 10 minutes). The pellets recovered in each case were resuspended and analyzed be protein gel electrophoresis. The virus partitioning profiles at pH 5 and pH 9 were comparable. The 4% w/v PEG effectively precipitated TMV with the majority (90-95%) of the HBcAg remaining in solution. By increasing the PEG concentration to 10% w/v, HBcAg aggregated and was recovered by precipitation at a purity of 85-90%, with TMV being the sole impurity.

For comparison, the differential PEG precipitation was performed on a clarified extract (Condition A material), prior to activated carbon treatment. The clarified extract was adjusted to 4% w/v PEG and following a 1 hour incubation at 4° C., centrifuged at 10,000×g for 10 minutes. The pellet obtained was resuspended and sampled. Additional PEG was added to the supernatant, to bring the final concentration to 10% w/v. Following a 16 hour storage at 4° C., a 10,000×g for 10 minute centrifugation was performed and the pellet resuspended and sampled. Protein gel electrophoresis indicated that >95% of the TMV was precipitated with 4% w/v PEG with minimal levels of host protein impurities or HBcAg present. With the higher PEG concentration treatment, HBcAg was precipitated, however, so too were significant quantities of the host proteins that were also present in the clarified extract. This highlights the requirement for the activated carbon treatment, prior to differential centrifugation, in the purification of HBcAg VLPs.

For the removal of residual TMV from the 10% PEG precipitated HBcAg, chromatography using hydroxyapatite resin (Macroprep Ceramic Type 1 80, BioRad, Hercules, CA) was performed. The material was initially dialyzed into the equilibration buffer (10 mM potassium phophate/138 mM NaCl, pH 7.4) and following capture by the resin, bound protein was eluted from the column using a linear gradient with a maximum potassium phosphate concentration of 500 mM (with pH and NaCl concentration maintained constant). Under these conditions the residual TMV and HBcAg were effectively separated.

### Example 7

### Activated Carbon As a Means for Purifying Icosahedral Viruses and Virus-like Particles: Brome Mosaic Virus

To evaluate the generalizability of the activated carbon procedure, with regard to icosahedral viruses and virus-like particles , it was further evaluated with brome mosaic virus, a 27 nm icosahedral virus with T=3 symmetry. N. benthamiana plants were inoculated with either the BMV expressing TMV-vector or an empty vector to serve as a control. Infected tissue from both sets of plants, together with uninoculated N. benthamiana tissue was harvested and homogenized in a Waring blender or using a pestle and mortar, with three volumes of chilled water containing 0.4% w/v sodium metabisulfite. The homogenate was passed through four layers of cheesecloth and the resulting “green juice” (GJ) extract was centrifuged at 10,000×g for 10 minutes. The clarified extract was adjusted to pH 9 and the precipitate that formed during pH adjustment removed by an additional 10,000×g/10 minute centrifugation. The supernatants from this second centrifugation were divided and contacted with activated carbon (Grade KBFF, Norit), at 1% w/v and 5% w/v, for 1 hour at 4° C. with mixing. Activated carbon was removed by centrifugation at 2000×g for 30 minutes and the recovered supernatants were analyzed by protein gel electrophoresis on a 10-20% trig glycine gel, and proteins were visualized by Coomassie blue staining.

For the BMV containing GJ extracts, a prominent 21 kDa band was visible, corresponding to the BMV coat protein. This band was absent in the empty. TMV vector control. The TMV coat protein band migrated at 17-18 kDa and TMV accumulation was approximately 3-fold higher than BMV, based on band staining intensity. Plant host proteins were also evident. Following treatment with 1% w/v activated carbon, there was only a minor reduction in host protein levels in the supernatant. However, when the activated carbon concentration was increased to 5% w/v, there was a substantial reduction in soluble plant proteins in the supernatant, with proteins across the whole molecular weight range (less than 6 kDa, to 220 kDa) being effectively adsorbed by the activated carbon. The 21 kDa BMV and ˜18 kDa TMV coat protein bands remained in the supernatant. These proteins were excluded from the pores of the activated carbon particles, by virtue of the virus dimensions. This example together with the results for the HBcAg VLPs, illustrates the application of activated carbon in the purification of icosahedral particles.

### Example 8

### Construction Of Vectors Permitting the Insertion Of Epitopes Into the Coat Protein Through Oligonucleotide Annealing

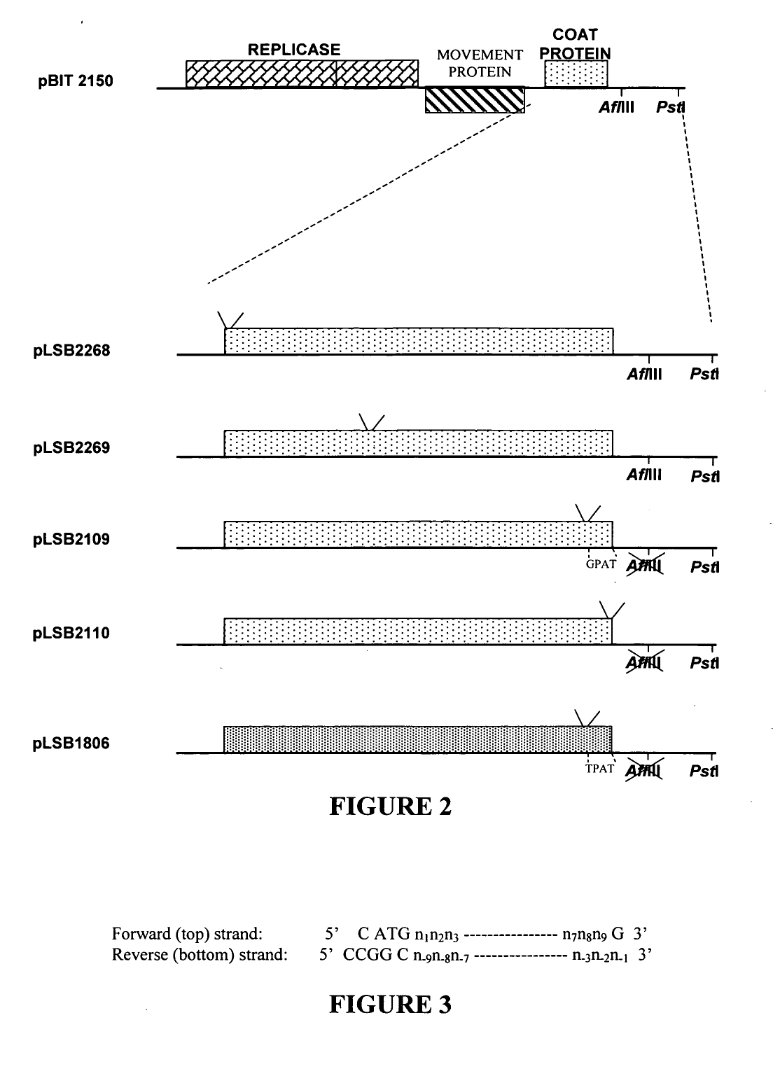

The cloning of various epitopes into different locations of the TMV coat protein was simplified by creating five acceptor vectors (FIG. 2). Table 12 lists these vectors along with their properties. These vectors contain the NcoI (5′ ) and NgoMIV (3′ ) restriction sites that were placed at the appropriate location of the coat protein open reading frame (ORF) for the TMV U1or U5 strain. For the TMV U1 strain, the restriction site pair was placed at the N-terminal, Loop, between amino acids 155 and 156 (GPAT) and at the C-terminus, while for the U5 strain it was only placed between amino acids 155 and 156 (TPAT). Any pair of oligonucleotides coding for a peptide, having the 5′ and 3′ overhangs of “CATG” and “CCGG”, respectively, can easily be cloned into these acceptor vectors. The construction of three of these vectors, pLSB2268, pLSB2269, and pLSB2109 was described by Palmer et al. (April 2004; world patent publication no. WO 2004/032622 A2). The construction of the remaining two vectors, pLSB2110 and pLSB1806, is described below.

TABLE 12
Characteristics of acceptor vectors used for inserting DJ5 epitopes
into TMV coat protein. The additional non-native sequence is the
amino acids generated at the insertion site.

| Plasmid | Insert | Source of | Added non-native | |
|---|---|---|---|---|
| name | location | coat protein | sequence | SEQ ID |
| pLSB2268 | N-terminal | U1 | ---AG | * |
| pLSB2269 | Loop | U1 | GSPM---AGPSG | * |
| pLSB2109 | Before the | U1 | AM---A | * |
| | last 4 amino | | | |
| | acids (GPAT) | | | |
| pLSB2110 | C-terminal | U1 | AM---AG | 8S |
| pLSB1806 | Before the | U5 | AM---AG | 9S |
| | last 4 amino | | | |
| | acids (TPAT) | | | |

The DJ5 peptide sequence is represented by the dashed line.
* More detailed information on this vector is available in Palmer et al. (April 2004; world patent publication no. WO 2004/032622 A2).
The nucleic acid sequences for pLSB2110 and pLSB1806 are for the coat protein open reading frame.

To generate pLSB2110, a 0.8 kilobase (kb) fragment of DNA was amplified from plasmid pLSB2108, a derivative of pBIT 2150 (Pogue et al, 2004; U.S. Pat. No. 6,730,306 B1) where the AflIII restriction site was removed, using the following primers:

SEQ ID 1S: GCGCACATGTCTTACAGTATCACTAC
SEQ ID 2S: TGGTCCTGCAACTGCCATGGACAGTGCCGGCTGAGGTAG
TCAAGAT
SEQ ID 3S: CGGATAACAATTTCACACAGGA

The SEQ ID 1S primer (AflIII 5′ coat), contains the starting sequence of the U1 coat protein and the AflIII recognition site is underlined. In the case of the SEQ ID 2S primer (NcoI/NgoMIV loopout at end), the NcoI and NgoMIV recognition sites are underlined. The SEQ ID 3S oligonucleotide (30B 7792R) anneals just downstream of the PstI site. The resulting product from this polymerase chain reaction (PCR) contained the coat protein that was modified at the C-terminus to provide two cloning sites, NcoI and NgoMIV, and the 3′ untranslated region (UTR) of the virus. This 0.8 kb AflIII PstI fragment was inserted into the 8.4 kb NcoI/ PstI fragment of vector pBIT 2150. The resulting plasmid, pLSB2110, allows the insertion of any peptide sequence, possessing both NcoI and NgoMIV overhangs, at the C-terminus of the U1 coat protein.

To generate pLSB1806, overlapping PCR was employed. Two DNA fragments, 0.5 kb and 0.3 kb in size, were amplified using plasmid BSG1057 as a template (Fitzmaurice et al., U.S. Pat. No.: 6,656,726 B1). The 0.5 kb fragment was amplified using oligonucleotides Afl-U5-F (SEQ ID 4S; CCACATGTATACAATCAACTCTCCGAG) and U5-NN-TPAT-R (SEQ ID 5S; CACTGTCCATGGCTGTGGTCC). This resulting fragment contained most of the U5 coat protein (amino acid no. 1-155), however, it lacks the second amino acid residue (proline). The 0.3 kb fragment was amplified using oligonucleotides U5-NN-TPAT (SEQ ID 6S; CTTGTCTGGACCACAGCCATGGACAGTGCCGGCACTCCG

GCTACTTAG) and JAL302 (SEQ ID 7S; AAACATGATTACGCCAAGCTTGCATG). This fragment contains the C-terminal four amino acids of the U5 coat protein as well as the 3′ UTR. In addition, it also possesses the two cloning sites, NcoI and NgoMIV, that were placed between amino acid number 155 and 156. Both 0.5 and 0.3 kb fragments were purified to remove all remaining oligonucleotides. These purified DNA fragments were mixed together and amplified by PCR using the two outermost oligonucleotides, AflIII and JAL302. The resultant 0.8 kb AflIII/ PstI fragment was subsequently cloned into the 8.4 kb NcoI/ PstI fragment from plasmid pBIT2150. The resulting plasmid pLSB1806 allows the insertion of any peptide sequence, possessing both NcoI and NgoMIV overhangs, at position 155 of the U5 coat protein (before the last four amino acids).

### Example 9

### Construction Of Coat Protein Fusions Displaying the 20 Amino Acid DJ5 Peptide

The initial five DJ5 coat protein fusion constructs are summarized in Table 13. Four of these constructs employed the U1 strain of TMV. The 20 amino acid DJ5 peptide (VHQANPRGSAGPCCTPTKMS; SEQ ID 10S) was fused on the surface exposed N and C terminus of the coat protein as well as within the surface exposed “60s” loop between amino acids 64 (Pro) and 67 (Asp), with the concomitant deletion of amino acids 65 (Asp) and 66 (Ser). In the final U1 strain fusion, the epitope was placed internal to the coat protein C-terminal four amino acids (the GPAT position). For the one U5 strain coat protein. fusion, the epitope was also placed internal to the C-terminal four amino acids (the TPAT position).

TABLE 13
Coat protein fusion vectors used to express recombinant virions
displaying the 20 amino acid DJ5 peptide in planta.

| Vector | Shorthand | Coat protein | Epitope insertion |
|---|---|---|---|
| Designation | descriptor | backbone | location |
| pLSB2655 | DJ5(20)-U1-GPAT | strain U1 | GPAT position |
| pLSB2656 | DJ5(20)-U1-C | strain U1 | C-terminus |
| pLSB2657 | DJ5(20)-U1-N | strain U1 | N-terminus |
| pLSB2658 | DJ5(20)-U1-L | strain U1 | surface exposed “60s” |
| | | | loop |
| pLSB2659 | DJ5(20)-U5-TPAT | strain U5 | TPAT position |

To introduce the 20 amino acid DJ5 epitope into these 5 locations, i.e. into the five plasmids described in Example 8, a set of oligonucleotides was designed as illustrated in FIG. 3. The actual sequences of the two oligonucleotides employed, together with associated SEQ IDs, are shown in Table 14.

TABLE 14

Forward and reverse oligonucleotides employed
in the cloning of the 20 amino acid DJ5 peptide
coat protein fusions, to yield plasmids
pLSB2655, pLSB2656, pLSB2657, pLSB2658
and pLSB2659.

| | |
|---|---|
| Forward oligonucleotide | Reverse oligonucleotide |

| | SEQ | | SEQ |
|---|---|---|---|
| Nucleic acid sequence | ID | Nucleic acid sequence | ID |
| CATGGTTCATCAAGCTAAT | 12S | CCGGCAGACATCTTAGTT | 13S |
| CCAGAGGATCTGCTGGACC | | GGAGTACAACATGGTCCA | |
| ATGTTGTACTCCAACTAAG | | GCAGATCCTCTTGGATTA | |
| ATGTCTG | | GCTTGATGAAC | |

To anneal the forward and the reverse oligonucleotides, 100 pmoles of each oliognucleotide was combined in 10× PCR buffer (Promega) and adjusted to a final volume of 20 uL with water. The oligonucleotide mix was heated to 95° C. for 3 minutes and subsequently cooled gradually to 30° C. at a rate of 0.1° C. per second. The reaction was held at 30° C. and 80 uL of water was added to each tube. For each ligation reaction, 1 uL of the annealed oliognucleotide mix (containing 1 pmole of each oligonucleotide) was employed and combined with 40 ng of the plasmid or vector of interest (cut with the NcoI and NgomIV restriction enzymes (both New England Biolabs)), together with 5 uL of 2× Quick ligation buffer (New England Biolabs) and 0.5 uL of Quick Ligase (New England biolabs). The ligation reaction volume was adjusted to 10 uL and following a 5 minute incubation at room temperature, 2 uL of the reaction was transferred to a 1.5 mL microfuge tube and chilled on ice. To this microfuge tube, 40 uL DH5a competent cells (Invitrogen) were added and the cell/ligation reaction mixture was incubated on ice for 30 minutes. The cells were then heat shocked at 37° C. for 2 minutes and the microfuge tube immediately returned to the ice. 950 uL of SOC medium was added to the microfuge tube, which was capped and shaken horizontally at 200 rpm and 37° C. for 1 hour. The cells were plated on Luria broth (LB) agar plates (50 or 100 uL per plate), containing 100 mg/mL ampicillin, and incubated overnight at 37° C. Single colonies were selected and 2 mL overnight cultures were grown in LB media containing 100 mg/mL ampicillin. The plasmid was purified from the DH5a cells and sequenced to confirm the presence of the 20 amino acid DJ5 epitope sequence. The correspondence between the starting vectors and the final vectors containing the 20 amino acid DJ5 epitope at the various insertion sites is summarized in Table 15, together with the SEQ IDs for the DJ5 epitope containing plasmids. Table 16 gives the final amino acid sequences of the translated coat protein fusions displaying the 20 amino acid DJ5 peptide and their associated SEQ IDs

TABLE 15
Correspondence between the initial cloning vector and the final
vector containing the 20 amino acid DJ5 peptide sequence.

| | |
|---|---|
| Cloning | |
| vector | DJ5 Vector |

| Designation | Designation | SEQ ID | Shorthand descriptor |
|---|---|---|---|
| pLSB2109 | pLSB2655 | 14S | DJ5(20)-U1-GPAT |
| pLSB2110 | pLSB2656 | 15S | DJ5(20)-U1-C |
| pLSB2268 | pLSB2657 | 16S | DJ5(20)-U1-N |
| pLSB2269 | pLSB2658 | 17S | DJ5(20)-U1-L |
| pLSB1806 | pLSB2659 | 18S | DJ5(20)-U5-TPAT |

TABLE 16

Full amino acid sequence of the 20 amino acid
DJ5 peptide coat protein fusion, together with
their associated SEQ IDs.

| Designation | | Coat protein amino |
|---|---|---|
| Shorthand | SEQ | acid sequence (inserted |
| descriptor | ID | amino acids are underlined) |
| pLSB2655 | 19S | MSYSITTPSQFVFLSSAWADPIELINLCTNALGN |
| DJ5(20)-U1- | | QFQTQQARTVVQRQFSEVWKPSPQVTVRFPDSDF |
| GPAT | | KVYRYNAVLDPLVTALLGAFDTRNRIIEVENQAN |
| | | PTTAETLDATRRVDDATVAIRSAINNLIVELIRG |
| | | TGSYNRSSFESSSGLVWTSAMVHQANPRGSAGPC |
| | | CTPTKMSAGPAT |
| pLSB2656 | 20S | SYSITTPSQFVFLSSAWADPIELINLCTNALGNQ |
| DJ5(20)-U1-C | | FQTQQARTVVQRQFSEVWKPSPQVTVRFPDSDFK |
| | | VYRYNAVLDPLVTALLGAFDTRNRIIEVENQANP |
| | | TTAETLDATRRVDDATVAIRSAINNLIVELIRGT |
| | | GSYNRSSFESSSGLVWTSGPATAMVHQANPRGSA |
| | | GPCCTPTKMSAG |
| pLSB2657 | 21S | MVHQANPRGSAGPCCTPTKMSAGSYSITTPSQFV |
| DJ5(20)-U1-N | | FLSSAWADPIELINLCTNALGNQFQTQQARTVVQ |
| | | RQFSEVWKPSPQVTVRFPDSDFKVYRYNAVLDPL |
| | | VTALLGAFDTRNRIIEVENQANPTTAETLDATRR |
| | | VDDATVAIRSAINNLIVELIRGTGSYNRSSFESS |
| | | SGLVWTSGPAT |
| pLSB2658 | 22S | MSYSITTPSQFVFLSSAWADPIELINLCTNALGN |
| DJ5(20)-U1-L | | QFQTQQARTVVQRQFSEVWKPSPQVTVRFPGSPM |
| | | VHQANPRGSAGPCCTPTKMSAGPSGDFKVYRYNA |
| | | VLDPLVTALLGAFDTRNRIIEVENQANPTTAETL |
| | | DATRRVDDATVAIRSAINNLIVELIRGTGSYNRS |
| | | SFESSSGLVWTSGPAT |
| pLSB2659 | 23S | MYTINSPSQFVYLSSAYADPVQLINLCTNALGNQ |
| DJ5(20)-U5- | | FQTQQARTTVQQQFADAWKPVPSMTVRFPASDFY |
| TPAT | | VYRYNSTLDPLITALLNSFDTRNRIIEVDNQPAP |
| | | NTTEIVNATQRVDDATVAIRASINNLANELVRGT |
| | | GMFNQASFETASGLVWTTAMVHQANPRGSAGPCC |
| | | TPTKMSAGTPAT |

### Example 10

### Construction Of Coat Protein Fusions Displaying the 12 Amino Acid N-terminal Region Of the DJ5 Peptide

Two additional DJ5-derived coat protein fusion constructs are summarized in Table 17, both of which employed the U1 strain of TMV. The fusions displayed the 12 amino acid N-terminal region of the DJ5 peptide (VHQANPRGSAGP; SEQ ID 11 S) fused to either the surface exposed N terminus of the coat protein or placed internal to the coat protein C-terminal four amino acids (the GPAT position).

TABLE 17
Coat protein fusion vectors used to express recombinant
virions displaying the 12 amino acid N-terminal region
of the DJ5 peptide in planta.

| | Shorthand | Coat protein | Epitope insertion |
|---|---|---|---|
| Designation | descriptor | backbone | location |
| pLSB2663 | DJ5(12)-U1-N | strain U1 | N-terminus |
| pLSB2664 | DJ5(12)-U1-GPAT | strain U1 | GPAT position |

To introduce the 12 amino acid epitope into these 2 locations, a set of two oligonucleotides was designed, the sequences of which are shown in Table 18, together with their associated SEQ IDs. To anneal the forward and the reverse oligonucleotides, the procedure outlined in Example 9 was followed and for each ligation reaction 1 uL of the annealed oliognucleotide mix (containing 1 pmole of each oligonucleotide) was employed. This was combined with the plasmid of interest (cut with the NcoI and NgomIV restriction enzymes) and the ligation reaction protocol together with its transformation into chemically competent DH5a cells was as detailed in Example 9.

TABLE 18

Forward and reverse primers employed in the
cloning of the coat protein fusions consisting
of the 12 amino acid N-terminal region of the
DJ5 peptide, to yield plasmids pLSB 2663 and
pLSB 2664.

| | |
|---|---|
| Forward primer | Reverse primer |

| Nucleic acid | SEQ | Nucleic acid | SEQ |
|---|---|---|---|
| sequence | ID | sequence | ID |
| CATGGTTCATCAA | 24S | CCGGCTGGTCCA | 25S |
| GCTAATCCAAGAG | | GCAGATCCTCTT | |
| GATCTGCTGGACC | | GGATTAGCTTGA | |
| AG | | TGAAC | |

The cells were plated on LB agar plates (50 or 100 uL per plate), containing 100 mg/m ampicillin, and incubated overnight at 37° C. Single colonies were selected and 2 mL overnight cultures were grown in LB media containing 100 mg/mL ampicillin. The plasmid was purified from the DH5a cells and sequenced to confirm the presence of the 12 amino acid DJ5-derived epitope sequence. The correspondence between the starting vectors and the final vectors containing the 12 amino acid DJ5-derived epitope at the two chosen insertion sites is summarized in Table 19, together with the SEQ IDs for the DJ5 epitope containing plasmids. Table 20 gives the final amino acid sequences of the translated coat protein fusions displaying the 12 amino acid N-terminal region of the DJ5 peptide and their associated SEQ IDs.

TABLE 19
Correspondence between the initial cloning vector and the final vector
containing the 12 amino acid DJ5-derived peptide sequence.

| | |
|---|---|
| Cloning | |
| vector | DJ5 Vector |

| Designation | Designation | SEQ ID | Shorthand descriptor |
|---|---|---|---|
| pLSB2268 | pLSB2663 | 26S | DJ5(12)-U1-GPAT |
| pLSB2109 | pLSB2664 | 27S | DJ5(12)-U1-C |

TABLE 20

Full amino acid sequence of the coat protein
fusions displaying the 12 amino acid N-terminal
region of the DJ5 peptide, together with their
associated SEQ IDs.

| Designation | | Amino acid sequence |
|---|---|---|
| Shorthand | SEQ | (inserted amino |
| descriptor | ID | acids are underlined) |
| pLSB2663 | 28S | MVHQANPRGSAGPAGSYSITTPSQFVFLSSAWAD |
| DJ5(12)-U1-N | | PIELINLCTNALGNQFQTQQARTVVQRQFSEVWK |
| | | PSPQVTVRFPDSDFKVYRYNAVLDPLVTALLGAF |
| | | DTRNRIIEVENQANPTTAETLDATRRVDDATVAI |
| | | RSAINNLIVELIRGTGSYNRSSFESSSGLVWTSG |
| | | PAT |
| pLSB2664 | 29S | MSYSITTPSQFVFLSSAWADPIELINLCTNALGN |
| DJ5(12)-U1- | | QFQTQQARTVVQRQFSEVWKPSPQVTVRFPDSDF |
| GPAT | | KVYRYNAVLDPLVTALLGAFDTRNRIIEVENQAN |
| | | PTTAETLDATRRVDDATVAIRSAINNLIVELIRG |
| | | TGSYNRSSFESSSGLVWTSAMVHQANPRGSAGPA |
| | | GPAT |

### Example 11

### Production Of TMV 2655, TMV 2656, TMV 2657, TMV 2658 and TMV 2659

The virus TMV 2655 was produced by transcription of plasmid pLSB 2655. Infectious transcripts were synthesized from transcription reactions with T7 RNA polymerase (Ambion) according to the manufacturers instructions. Following the verification of transcript integrity by agarose gel electrophoresis, the RNA transcript was combined with an abrasive solution (a benonite/celite mixture suspended in a glycine/phosphate buffer containing sodium pyrophosphate ) and used to inoculate Nicotiana benthamiana leaves of 23 to 28 day old plants. Approximately 5 to 13 days post-inoculation, depending on the severity of the infection, systemic movement of the recombinant virus was visible in the plant tissue, by virtue of a mosaic phenotype on the virus-containing leaves. Systemically infected tissue was harvested for virus. extraction and purification. It should be noted that alternative host plants, other than Nicotiana benthamiana can be employed in the production of TMV 2655. For example, Nicotiana excelsiana or Nicotiana tabacum represent two possible alternative plant hosts. For the latter two hosts, tissue is harvested 2.5-5 weeks post inoculation, after systemic spread of the virus.To produce TMV 2656 virus, transcript was generated from plamsid pLSB2656, inoculated onto plants and systemically infected tissue harvested in a manner similar to that described for the production of virus TMV 2655.To produce TMV 2657 virus, transcript was generated from plamsid pLSB2657, inoculated onto plants and systemically infected tissue harvested in a manner similar to that described for the production of virus TMV 2655.To produce TMV 2658. virus, transcript was generated from plamsid pLSB2658, inoculated onto plants and systemically infected tissue harvested in a manner similar to that described for the production of virus TMV 2655.To produce TMV 2659 virus, transcript was generated from plamsid pLSB2659, inoculated onto plants and systemically infected tissue harvested in a manner similar to that described for the production of virus TMV 2655.

### Example 12

### Extraction and Purification Of TMV 2655, TMV 2656. TMV 2657, TMV 2658 and TMV 2659

The recombinant virus TMV can be extracted from the infected plant tissue immediately following harvesting. Alternatively, the tissue can be can be stored for up to 14 days at 4° C., or at −20° C. to −80° C. (for days to months) prior to performing the extraction. The tissue can also be flash frozen prior to extraction, to aid in tissue disintegration.

Several procedures have been documented for the purification of recombinant TMV virus from infected plant tissue. For examples Garger et al. (U.S. Pat. Nos. 6,033,895, 6,037,456 and 6,303,779) and Pogue et al. (U.S. Pat. No. 6,740,740) disclose methods based on the pH adjustment and heat treatment of the homogenate “green juice” obtained following extraction of the infected tissue. Pogue et al. also disclose a procedure based on the use of polyethyleneimine (PEI) to aid in the separation of the plant host proteins and the recombinant TMV. These procedures and modifications thereof, designed to improve epitope stability (i.e. minimize degradation by proteolysis) during extraction and processing,. and recombinant virion solubility, were used in the purification of virus TMV 2655, the purification of TMV 2656, the purification of TMV 2657, the purification of TMV 2658 and the purification of TMV 2659.

One of the extraction procedures employed in the case of TMV 2655 was as follows. Systemically infected plant tissue (leaf and stalks) was harvested and combined with chilled extraction buffer EB (100 mM Tris, pH 8, 0.86 M sodium chloride, 0.2% v/v Triton X-100), to which 0.04% w/v sodium metabisulfite had been added, at a buffer volume (mL) to tissue mass (g) ratio of 2:1. The plant tissue and extraction buffer were homogenized for 1 minute in a 1 L Waring blender, transferred to an Erlenmeyer flask and further homogenized for 1 minute using a Polytron (Brinkman Instruments). This homogenate was passed through four layers of cheesecloth, to remove the fiber to yield approximately 170 ml of plant extract, which will hereafter be referred to as green juice. The green juice was transferred to a centrifuge bottle, centrifuged at 10,000×G for 10 minutes and the supernatant discarded as the majority of TMV DJ5 coat protein fusion, which was insoluble, was present in the pellet. The pellet was resuspended in approximately 160 ml of the extraction buffer EB, with the aid of the Polytron (1 minute of homogenization). Following the Polytron treatment, the resuspended pellet was transferred to a centrifuge bottle, centrifuged at 10,000×G for 10 minutes and the supernatant discarded. This pellet resuspension in extraction buffer EB, Polytron homogenization and centrifugation at 10,000×G for 10 minutes was repeated a further two times. The purpose of these repeated steps was to effect the separation of the plant-derived proteins and pigments from the insoluble TMV DJ5 coat protein fusion, which was facilitated by the presence of a relatively high sodium chloride concentration and detergent in the buffer EB. The number of repetitions required to remove all the plant-derived pigments, to yield a white to light tan pellet, may be dependent on the age of the harvested tissue and the TMV coat protein fusion being expressed. If green host-derived pigment remains associated with the pellet, additional washes to the TMV coat protein fusion-containing pellet can be performed employing a high pH buffer, for example 50 mM triethylamine containing 0.2% v/v Triton X-100 and 0.04% w/v sodium metabisulfite (buffer B1). For TMV 2655, these additional pellet washes were performed. Specifically the pellet obtained following the three buffer EB washes was resuspended in 160 mL of buffer B1 with the aid of the Polytron (1 minute of homogenization) and then transferred to a centrifuge bottle, centrifuged at 10,000×G for 10 minutes and the supernatant discarded. This pellet was subjected to an additional buffer B1 wash and the pellet was then resuspended, with the aid of the Polytron, in approximately 160 mL of 1× phosphate buffered saline, pH 7.4, centrifuged at 10,000×G for 10 minutes and the supernatant discarded. A second similar PBS wash of the pellet was performed and the final pellet was resuspended in approximately 16 mL of 1× PBS. The purpose of the two 160 mL PBS washes was to remove residual detergent from the TMV DJ5 coat protein fusion-containing pellet and ensure that the final TMV coat protein fusion preparation was close to neutral pH. Aliquots of the green juice, the discarded supernatants and the final pellet preparation, resuspended in 1× PBS, were subjected to PAGE analysis. The PAGE analysis showed that the supernatants contained minimal amounts of the TMV 2655 DJ5 coat protein fusion, whereas this was the principal protein species present in the final pellet preparation. Conversely the majority of the plant-derived host proteins were present in the discarded supernatants, and minimal host protein was detected in the final pellet. The same procedure was employed in the purification of TMV 2656, the purification of TMV 2658 and the purification of TMV 2659, with similar results.

In the case of TMV 2657 the following procedure was employed. Systemically infected leaf and stalk tissue was macerated in a Waring blender for 1 minute at the high setting with chilled buffer EB1 (0.86 M sodium chloride, containing 0.04% w/v sodium metabisulfite) at a buffer (mL) to tissue (g) ratio of 2:1. The macerated material was strained through four layers of cheesecloth to remove fibrous material. The resultant green juice was adjusted to a pH of 5.0 with phosphoric acid. The pH adjusted green juice was heated to 47° C. and held at this temperature for 5 minutes and then cooled to 15° C. The heat-treated green juice was centrifuged at 6,000×G for 3 minutes resulting in two fractions, supernatant S1 and pellet P1. The pellet P1 fraction was resuspended in distilled water using a volume of water equivalent to 1/2 of the initial green juice volume. The resuspended pellet P1 was adjusted to a pH of 7.5 with sodium hydroxide and centrifuged at 6,000×G for 3 minutes resulting in two fractions, supernatant S2 and pellet P2. Virus was precipitated from both supernatant fractions S1 and S2 by the addition of 4% w/v polyethylene glycol (PEG) 6,000 and 4% w/v sodium chloride. After incubation at 4° C. (1 hour), precipitated virus was recovered by centrifugation at 10,000×G for 10 minutes. The virus pellet was resuspended in 1× PBS, pH 7.4 and clarified by centrifugation at 10,000×G for 3 minutes to yield a final clarified TMV 2657 preparation. Aliquots of the green juice, the supernatants S1 and S2 and the final virus preparation pre and post the clarification spin were subjected to PAGE analysis. The PAGE analysis showed the majority of the principal coat protein band present in the green juice partitioned into the supernatant S1 with low levels present in the supernatant S2. With PEG precipitation of the supernatant S1 and the supernatant S2 and the final clarification spins, virus was further purified from the plant host proteins to yield two substantially pure TMV 2657 virus preparations. The majority of the TMV 2657 virus recovered was present in the pellet obtained following the supernatant S1 PEG precipitation. A minor portion of the TMV 2657 virus was removed by the final clarification spin, together with residual plant host proteins.

It should be noted that the procedure outlined for TMV 2657 was applied to the other DJ5 epitope TMV coat protein fusions, namely TMV 2655, TMV 2656, TMV 2658 and TMV 2659. For TMV 2655, TMB 2656 and TMV 2658, PAGE analysis indicated that the coat protein band was present in the initial green juice, however the band was absent from both the supernatant S1 and the supernatant S2 and no TMV coat protein fusion was recovered by the procedure outlined for TMV 2657. Further analysis showed that TMV 2655, TMV 2656 and TMV 2658 were insoluble and present in the pellet P2, together with plant pigments and proteins. To purify the insoluble TMV 2655, TMV 2656 and TMV 2658 from the plant-derived proteins and pigments, the procedure outlined above for TMV 2655 was employed. In the case of TMV 2659, the procedure outlined for TMV 2657 was initially unsuccessful. When extractions of freshly harvested infected tissue were performed, employing a Waring blender for homogenization, minimal full length TMV 2659 was recovered, due to degradation that occurred during processing. By modifying the procedure and starting with frozen tissue that was processed with a mortar and pestle followed by Polytron homogenization, approximately 30-40% of full-length TMV 2659 was present in the supernatant S1. This was concentrated by PEG precipitation and 15-17% of this TMV 2659 remained soluble following the final clarification spin, with the remainder present in the clarification pellet. Both the clarification pellet and the clarified virus preparation contained significant quantities of plant host proteins, resulting in a final product with low purity. These results suggested that the starting tissue state (fresh vs. frozen) and/or the tissue disintegration step(s) employed played a role in epitope stability. Since TMV 2659 exhibited partial solubility, further optimization was performed on the TMV 2657 procedure, to determine if recovery and purity of the final TMV 2659 virus preparation could be improved. The final procedure employed was the following. Frozen, systemically infected leaf and stalk tissue was combined with 2 volumes of buffer EB1 and macerated with a pestle and mortar, followed by further homogenization using a Polytron. This extract was strained through four layers of cheesecloth and the resultant green juice was adjusted to a pH of 5.0 with phosphoric acid. The pH adjusted green juice was centrifuged at 6,000×G for 3 minutes resulting in two fractions, supernatant S1 and pellet P1, the latter of which was not processed further. The supernatant S1 was adjusted to pH 6 by the addition of sodium hydroxide and contacted with 5% w/v activated carbon powder (e.g. Nuchar grade SA-20 or Norit grade KB-FF) for 1 hour at 4° C. The activated carbon containing supernatant S1 was then adjusted to pH 8 with sodium hydroxide and centrifuged at 3000×G for 15 minutes to remove the activated carbon. The supernatant from this was taken forward and the TMV 2659 precipitated by the addition of 4% w/v polyethylene glycol (PEG) 6,000 and 4% w/v sodium chloride. After incubation at 4° C. (1 hour), precipitated virus was recovered by centrifugation at 10,000×G for 10 minutes. The virus pellet was resuspended in 1× PBS, pH 7.4 and no clarification spin was performed. Aliquots of the green juice, the supernatant S1 at the various stages of processing, the resuspended pellet P1 and the final TMV 2659 preparation were subjected to PAGE analysis. As noted previously, approximately 40% of the green juice coat protein was present in the supernatant S1 together with substantial levels of plant host proteins, while visually the majority of the green pigment partitioned into the pellet P1. Following the activated carbon treatment at pH 6 there was a substantial reduction in the host protein level in the supernatant with recovery of 70-80% of the TMV 2659. With pH 8 adjustment and centrifugation to remove the activated carbon the TMV 2659 losses were minimal. PEG precipitation from the pH 8 supernatant was performed to concentrate the TMV 2659, resulting in a final virus preparation with satisfactory purity and a notable improvement over the virus obtained from the procedure where no activated carbon or pH steps were employed.

The rationale behind the optimized procedure was the following. The initial adjustment to pH 5 was required to remove the plant pigments as well as the principal plant proteins, namely rubisco large and small subunit. Experience with other coat protein fusions has demonstrated that the plant extracts contain acidic protease(s), with optimal activity between pH 4.5 and pH 5.5 that can result in coat protein fusion degradation during processing. The supernatant S1 was therefore adjusted to pH 6. Activated carbon, by virtue of its complex pore structure effectively traps globular proteins while the TMV virion, owing to its dimensions, is excluded. By contacting the supernatant S1 with activated carbon the host proteins are adsorbed by the activated carbon and removed, improving the purity of the TMV 2659 in the supernatant S1. Furthermore, proteases are among the globular proteins adsorbed and coat protein fusion stability is therefore also improved. The activated carbon containing supernatant S1 was then adjusted to pH 8. This does not affect host proteins adsorbed to the activated carbon, but does result in the formation of a precipitate, the presence of which aids in the removal of the activated carbon with centrifugation, together with any remaining green pigment. In addition, TMV coat protein fusion solubility is generally improved under mild alkaline conditions. The PEG precipitation was performed to concentrate the virus and the final clarification spin omitted as this resulted in the precipitation of the partially soluble TMV 2659.

To arrive at this procedure a number of variations of the above were tested. For example no pH adjustment was performed on the initial green juice so that the initial pH was 5.5 to 5.6. This condition was tested as at higher pH, TMV fusion recovery in the supernatant S1 is generally improved. However, rubisco large and small subunit removal was incomplete and the rubisco complex is sufficiently large so as to be excluded from the activated carbon. As a result final purity was negatively impacted. Adjusting the green juice to pH 8 was also tested and following activated carbon treatment, the pH was adjusted to 5.0 to precipitate rubisco. Again, with this protocol, residual levels of the rubisco proteins were present in the final virus preparation.

Polyacrylamide gel electrophoresis (PAGE) analysis, and Western blot analysis (Table 21) were performed on the purified recombinant viruses to assess purity and epitope immunoreactivity. For the Western blot analysis a goat antibody raised against the pro-form of GDF-8 was employed. This antibody, denoted Goat #661, was determined to be neutralizing in an in vitro GDF-8 neutralization assay. Western blots were also performed with a rabbit antibody raised against wild-type TMV, denoted PVAS135D (obtained from the ATCC collection). The physical characteristics of the purified recombinant TMV fusions, i.e. solubility, are also noted in Table 21.

TABLE 21
Solubility, purity, polyacrylamide gel electrophoresis (PAGE) profile
and reactivity with the GDF-8 neutralizing Goat #661 antibody and the anti-TMV
antibody (PVAS-135D) by Western blot, for the 20 amino acid DJ5 peptide coat
protein fusions.

| Designation | | | | Western blot detection | |
|---|---|---|---|---|---|
| Shorthand descriptor | Solubility | Purity | PAGE profile | Anti-GDF-8 | Anti-TMV |
| TMV 2655 | Insoluble | >90% | Oligomeric ladder | Yes | Yes |
| DJ5(20)-U1-GPAT | | | (7 to 9 bands) | (5 to 6 bands) | (5 to 6 bands) |
| TMV 2656 | Insoluble | >90% | Oligomeric ladder | Yes | Yes |
| DJ5(20)-U1-C | | | (5 to 6 bands) | (5 to 6 bands) | (5 to 6 bands) |
| TMV 2657 | Soluble | >90% | Single band | No | Yes |
| DJ5(20)-U1-N | | | | | |
| TMV 2658 | Insoluble | >90% | Oligomeric ladder | Yes | Yes |
| DJ5(20)-U1-L | | | (5 to 6 bands) | (5 to 6 bands) | (5 to 6 bands) |
| TMV 2659 | Partially | >90% | Oligomeric ladder | Yes | Yes |
| DJ5(20)-U5-TPAT | soluble | | (5 to 6 bands) | (5 to 6 bands) | (5 to 6 bands) |

All the recombinant TMV fusions listed in Table 21 were successfully purified to greater than 90% purity. In the case of TMV 2655, TMV 2656 and TMV 2658, the purified TMV was insoluble. When analyzed by PAGE, a characteristic laddering pattern was observed for the three U1 strain fusions. On 10-20% Tris-glycine gels, the lowest (monomer) band migrated at approximately 22 kDa, as expected for the 20 amino acid DJ5 peptide coat protein fusion. The protein band above this monomer migrated at 45 kDa and the protein band above this at 65-70 kDa. By Western blot the majority of these bands were detected by the Goat #661 antibody as well as the anti-TMV PVAS-135D antibody (very high molecular weight bands, >200 kDa, were not always detected due to poor transfer from the gel to the membrane). Together with the observed molecular weights, these results indicate that the additional bands represent dimers, timers and higher multimers of the 20 amino acid DJ5 peptide coat protein fusion. When the PAGE analysis of TMV 2655, TMV 2656 and TMV 2658 was performed in the absence of reducing agent, the proportion of monomer decreased, with an observable increase in the proportion of higher order oligomers. This suggests that disulfide bridging between the 20 amino acid DJ5 peptide coat protein fusions was occurring. The 20 amino acid DJ5 peptide contains two cysteine residues, which are likely involved in the formation of the observed higher order oligomers. For TMV 2659, the final virus preparation was partially soluble and exhibited the same reducing agent-dependent oligomeric banding pattern as TMV 2655, TMV 2656 and TMV 2658 by both PAGE and Western blot analysis. The slightly improved solubility of TMV U5 may be due to the use of the strain U5 coat protein in place of the strain U1 coat protein. The only soluble TMV fusion from the series listed in Table 21 was TMV 2657, where the 20 amino acid DJ5 peptide was displayed as an N-terminal fusion to the strain U1 coat protein. For TMV 2657, the coat protein migrated with a mass of approximately 18 kDa on the PAGE gel, similar to the wild-type U1 coat protein. This suggested truncation of the epitope. No oligomeric ladder was observed and by Western blot the TMV fusion was detected by the anti-TMV PVAS135D antibody but not by the GDF-8 neutralizing Goat #661 antibody. This lack of reactivity with the Goat #661 antibody supports truncation of some or all of the 20 amino acid. DJ5 peptide fusion in the case of TMV 2657.

### Example 13

### Characterization Of TMV 2655, TMV 2656, TMV 2657, TMV 2658 and TMV 2659 By MALDI

In addition to PAGE and Western blot analysis, the virus preparations were characterized using Matrix Assisted Laser Desorption Ionization—Time of Flight (MALDI-TOF) (Table 11 T). PEG precipitated, resuspended virus preparations were diluted in a sinapinic acid (Aldrich, Milwaukee, Wis.) solution, with the dilution in the range of 1:1 to 1:20 depending in the virus concentration, to obtain a final concentration of 1 to 1.5 mg/mL. The sinapinic acid was prepared at a concentration of 10 mg/mL in 0.1% aqueous triflouroacetic acid/acetonitrile (70/30 by volume). The sinapinic acid treated sample (1.0 μl) was applied to a stainless steel MALDI plate surface and allowed to air dry at room temperature. MALDI-TOF mass spectra were obtained with a PerSeptive Biosystems DE-PRO (Houston, Tex.) operated in the linear mode. A pulsed laser operating at 337 nm was used in the delayed extraction mode for ionization. An acceleration voltage of 25 kV with a 90% grid voltage and a 0.1% guide wire voltage was used. Approximately 100 scans were acquired and averaged over the mass range 2,000-156,000 Da with a low mass gate of 2,000. Ion source and mirror pressures were approximately 1.2×10⁻⁷ and 1.6×10⁻⁷ Torr, respectively. All spectra were mass calibrated with a two-point fit using horse apomyoglobin (16,952 Da) and insulin (5734 Da) as standards.

TABLE 22
Summary of the expected and observed molecular weights,
by MALDI, for the 20 amino acid DJ5 peptide fusions.

| Designation | MALDI analysis | | |
|---|---|---|---|
| Shorthand descriptor | Expected MW | Observed MW | Match |
| TMV 2655 | 19,833 Da (-Met/ | 19,829 Da | Yes |
| DJ5(20)-U1-GPAT | Acetyl) | | |
| TMV 2656 | 19,890 Da (-Met/ | 19,890 Da | Yes |
| DJ5(20)-U1-C | Acetyl) | | |
| TMV 2657 | 19,685 (-Met/Acetyl) | 17,745 Da | No |
| DJ5(20)-U1-N | | | |
| TMV 2658 | 20,100 Da (-Met/ | 20,097 Da | Yes |
| DJ5(20)-U1-L | Acetyl) | | |
| TMV 2659 | 19,878 Da (-Met/ | 19,876 Da | Yes |
| DJ5(20)-U5-TPAT | Acetyl) | | |

For TMV 2655, TMV 2656, TMV 2658 and TMV 2659 the observed molecular weights matched the expected molecular weights, for the case where the coat protein fusion's N-terminal Met residue was removed and the adjacent amino acid acetylated. The presence of the intact 20 amino acid DJ5 epitope on TMV 2655, TMV 2656, TMV 2658 and TMV 2659, together with the positive anti-GDF-8 Western blot reported in Table 21, confirmed that all four of these TMV fusions were potential vaccine candidates. For the N-terminal fusion, TMV 2657, a mass of 17,745 Da was obtained, representing multiple truncation possibilities. By performing liquid chromatography on a tryptic digest of TMV 2657 and analyzing the resolved peptide fragments by tandem mass spectrometry it was determined that the C-terminus of TMV 2657 was intact and that the DJ5 epitope was cleaved, to leave only the final C-terminal serine, which was acetylated. Since this fusion failed to retain the 20 amino acid DJ5 epitope and was not detected in Western blots by the anti-GDF-8 Goat #661 antibody, it was not pursued further as a vaccine candidate. The confirmation by mass spectrometry that TMV 2657 lacked the DJ5 epitope and its migration as a single band by PAGE analysis (Table 21), indicates that amino acid residues within the DJ5 epitope were responsible for the cross-linking and higher order oligomer formation. As indicated above, the two cysteines within the DJ5 epitope were considered the most likely amino acids to be involved in this cross-linking.

### Example 14

### Extraction and Purification Of TMV 2663 and TMV 2664

Of the five 20 amino acid DJ5 peptide fusions purified (see Example 12), and characterized by mass spectrometry (Example 13), four were identified as potential vaccine candidates, namely TMV 2655, TMV 2656, TMV 2658 and TMV 2659. These four TMV fusions, however, were insoluble, although TMV 2659 could be purified in a partially soluble form. . Since the TMV fusion insolubility was correlated with the presence of the 20 amino acid DJ5 peptide, which resulted in coat protein cross-linking, it was hypothesized that the oligomer formation was related to the TMV fusion insolubility. Furthermore, the two cysteines present in the DJ5 peptide appeared responsible for this cross-linking, and so two new constructs were proposed in which these two residues were eliminated. The two new constructs, pLSB2663 and pLSB2664, were designed to display the N-terminal 12 amino acids of the DJ5 peptide, as N-terminal and GPAT fusions respectively, to the U1 coat protein.

The same points raised in Example 12, regarding tissue harvesting and storage prior to the extraction also apply to TMV 2663 and TMV 2664. As noted in Example 12, several procedures have been documented for the purification of recombinant TMV virus from infected plant tissue. These procedures and modifications thereof, designed to improve epitope stability (i.e. minimize degradation by proteolysis) during extraction and processing, and improve virion solubility, were used in the purification of virus TMV 2663 and the purification of TMV 2664.

In the case of TMV 2664, the procedure outlined for TMV 2657 in Example 12 was employed starting from freshly harvested, systemically infected plant tissue. PAGE analysis of the in process samples and the final clarified virus preparation showed that approximately 80% of the TMV 2664 present in the green juice partitioned into the supernatant S1, with the remainder present in the supernatant S2. Only the supernatant S1 was carried forward for PEG precipitation and following the final clarification spin, which precipitated some residual host proteins, the majority of the purified TMV 2664 remained soluble. When the procedure outlined for TMV 2657 in Example 12 was employed for the purification of TMV 2663, starting from freshly harvested tissue, only low levels of the product was present in the supernatant S1, with the majority of the TMV 2663 associated with the pellet P2. The protocol was modified such that the systemically infected tissue was frozen prior to extraction and tissue maceration was performed with the aid of a mortar and pestle. With these alterations, PAGE analysis indicated that approximately 30% of the TMV 2663 partitioned into the supernatant S1, with the remainder detected in the supernatant S2 and pellet P2. These results suggest that the starting tissue state (fresh vs. frozen) and/or the tissue disintegration step(s) employed play a role in recombinant virion solubility, possibly be reducing the association between the recombinant virion and the host plant proteins. The supernatant S1 was PEG precipitated and the concentrated virus subjected to a clarifying spin. The TMV 2663 partitioned into the clarification spin pellet and by PAGE showed minimal contamination by plant host proteins.

Polyacrylamide gel electrophoresis (PAGE) analysis, and Western blot analysis was performed on the purified recombinant viruses to assess purity. For the Western blot analysis a goat antibody raised against the pro-form of GDF-8 was employed. This antibody, denoted Goat #661, was determined to be neutralizing in an in vitro GDF-8 neutralization assay. Western blots were also performed with a rabbit antibody raised against wild-type TMV, denoted PVAS135D (obtained from the ATCC collection). The physical characteristics of the purified recombinant TMV fusions, i.e. solubility, are also noted in Table 23.

TABLE 23
Solubility, purity, polyacrylamide gel electrophoresis (PAGE) profile
and reactivity with the GDF-8 neutralizing Goat #661 antibody and
the anti-TMV antibody (PVAS-135D) by Western blot, for the
shortened 12 amino acid DJ5 peptide coat protein fusions.

| | | | | Western | |
|---|---|---|---|---|---|
| Designation | | | | blot detection | |
| Shorthand | | | PAGE | Anti- | |
| descriptor | Solubility | Purity | profile | GDF-8 | Anti-TMV |
| TMV 2663 | Partially | >90% | Single band | Yes | Yes |
| DJ5(12)-U1-N | soluble | | | | |
| TMV 2664 | Soluble | >90% | Single band | Yes | Yes |
| DJ5(12)-U1-N | | | | | |

Both recombinant TMV fusions listed in Table 23 were successfully purified to greater than 90% purity. In the case of TMV 2663, the final purified virus was partially soluble, while TMV 2664 was completely soluble. When analyzed by PAGE, both TMV fusions migrated as a single band and these coat protein fusions were detected by both the anti-TMV PVAS-135 and the anti-GDF-8 Goat #661 antibodies. Minimal or no higher molecular weight species were detected by PAGE or Western blot, supporting the hypothesized role played by the two DJ5 epitope cysteines in coat protein cross-linking. The improved solubility observed also indicates that the oligomerization was responsible for the macromolecular association of the recombinant TMV virions.

### Example 15

### Characterization Of TMV 2663 and TMV 2664 by MALDI

In addition to PAGE and Western blot analysis, the TMV 2663 and TMV 2664 virus preparations were characterized using Matrix Assisted Laser Desorption Ionization—Time of Flight (MALDI-TOF) (Table 24). The preparation and spotting of the PEG precipitated and resuspended virus in sinapinic acid was as outlined in Example 13. MALDI-TOF spectra acquisition on a PerSeptive Biosystems DE-PRO (Houston, Tex.) was also performed as described in Example 13, using horse apomyoglobin and insulin as mass standards.

TABLE 24
Summary of the expected and observed molecular weights, by
MALDI, for the shortened 12 amino acid DJ5 peptide fusions.

| Designation | MALDI analysis | | |
|---|---|---|---|
| Shorthand descriptor | Expected MW | Observed MW | Match |
| TMV 2663 | 18,794 Da (-Met/ | 18,792 Da | Yes |
| DJ5(12)-U1-N | Acetyl) | | |
| TMV 2664 | 18,981 Da (-Met/ | 18,977 Da | Yes |
| DJ5(12)-U1-GPAT | Acetyl) | | |

For both TMV 2663 and TMV 2664, the observed molecular weights matched the expected molecular weights, for the case where the coat protein fusion's N-terminal Met residue was removed and the adjacent amino acid acetylated. The presence of the intact 12 amino acid DJ5 epitope on TMV 2663 and TMV 2664, together with the positive anti-GDF-8 Western blot data reported in Table 23, confirmed that both of these TMV fusions were potential vaccine candidates.

### Example 16

### Application Of Polyethylenimine (PEI) to the Purification Of TMV 2663, TMV 2659 and their Goat Analog Sequences

The DJ5 region of GDF-8 is homologous for humans, swine, cattle and chickens. In the case of goats there is one conservative amino acid substitution at position 7 of the peptide, from an arginine to a lysine. Viral constructs of the goat version of vectors TMV 2663 and TMV 2659, denoted TMV U1827 and TMV U1826 respectively were prepared, following the procedures outlined in Example 9. Table 25 compares amino acid sequences of the original TMV vectors and their goat analogs.

TABLE 25

Comparison of the original DJ5 coat protein
fusions and their goat analog amino acid
sequences.

| | | |
|---|---|---|
| Abbreviation | pLSB # | Sequence |

| | | |
|---|---|---|
| DJ5(12) U1 | 2663 | VHQANPRGSAGP::U1 |
| G5(12) U1 | 1827 | VHQANPKGSAGP::U1 |
| DJ5(20) U5 | 2659 | U5::VHQANPRGSAGPCCTPTKMS GPAT |
| G5(20) U5 | 1826 | U5::VHQANPKGSAGPCCTPTKMS GPAT |

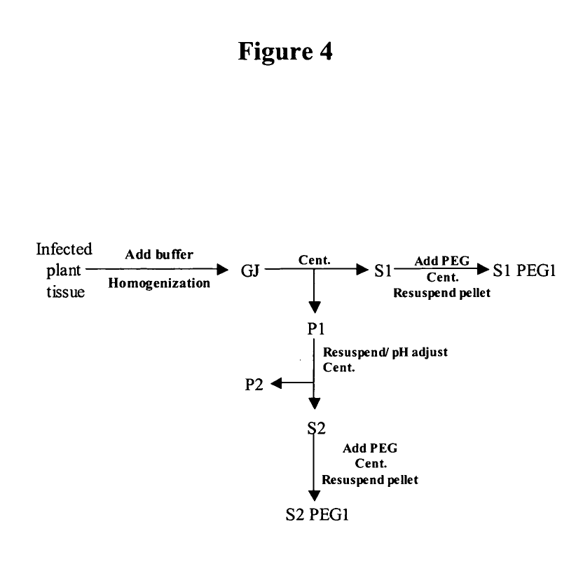

The process optimization outlined below was designed to effectively process larger masses of tissue and to recover final virus displaying the DJ5 or G5 epitopes at higher protein concentrations (4-5 mg/ml compared to 0.5-1 mg/ml) than the procedures outlined in Example 12. Since the amino acid substitution between the constructs was conservative in nature (Lys to Arg), process optimization was completed with TMV 2663 and the new procedure then evaluated for the goat analog construct. FIG. 4 provides a general outline of the nomenclature employed for the processing streams.

Extractions were performed from frozen tissue, as the preliminary processing studies (Example 12) indicated that fusion recovery was improved compared with fresh tissue homogenization. Extraction was performed either in the presence or absence of sodium chloride, with green juice (GJ) adjustment to pH 5 to promote rubisco precipitation, prior to the initial clarification spin to obtain the S1 supernatant. The pellet P1 was resuspended and adjusted to pH 7.5, to obtain the S2 and determine virus recovery under mildly alkaline conditions. In the absence of salt, no virus was present in either the S1 or the S2. With salt incorporation, approximately 60% of the virus partitioned into the S1, confirming that NaCl was required during extraction. The virus recoveries were comparable for extraction from either fresh or frozen tissue, indicating that differences in the method and/or duration of homogenization, owing to the larger tissue masses processed, influenced virus yield from the infected tissue. Fresh tissue was therefore employed for all subsequent experiments.

Processes were generally based on the pH 5 procedure, with salt present during extraction. The incorporation of a heat treatment step on the green juice was evaluated by gel electrophoresis. Purity was improved with the removal of residual levels of rubisco from the final virus preparation. Virus recovery in the S1 was not affected by the incorporation of the heat treatment step and recoveries following PEG precipitation were unchanged at 40-45%, with virus purity estimated at ˜85% to 90%.

At the higher protein concentrations attained for the final virus, green pigmentation of the polyethylene glycol (PEG) precipitated virus (S1 PEG 1) was visible, even when virus purity by SDS-PAGE was 90% or above. This pigment association was a characteristic of the DJ5 epitope, since the wild-type U1 virus at comparable concentrations was cream/white in appearance. Several different approaches were tested to address this pigmentation.

Incorporation of bentonite into the extraction procedure was tested. Bentonite, a magnesium aluminum silicate with a net negative charge, forms a colloidal structure when hydrated that is capable of trapping plant-derived components. With unrelated coat protein fusions, processing with bentonite improved most protein and pigment partitioning into the P1, eliminating green pigment association with the final purified virus. The TMV is thought to be excluded from the bentonite owing to its rod-like structure. However, for all the pH and salt combinations tested no DJ5(12) U1 virus was recovered in the S1. The DJ5(12) epitope confers a positive charge to the virus surface, which may promote association with the negatively charged bentonite. The addition of butanol to the green juice homogenate (6-8% w/v) was also tested. Butanol has previously been known to facilitate the coagulation of chloroplasts and their P1 partitioning. Relative to the control extractions lacking butanol addition, no reduction in the level of final virus pigmentation was observed.

The DJ5 peptides contribute a net positive charge to the capsid surface. Typically, previously displayed epitopes were either charge balanced or possessed a net negative charge. It was hypothesized that the surface exposed amine groups of TMV 2663 resulted in increased interactions with anionic host components and this was responsible for the increased pigmentation of the purified virus. Extractions were performed where Tris[hydroxymethyl]aminomethane was incorporated during tissue homogenization to determine if competition from a free amine would prevent pigment/virion interactions and increase virus recovery. The presence of Tris resulted in a modest (5-7%) increase in the virus recovered in the S1 supernatant, but the level of pigmentation of the final virus was unchanged. The failure of the free amine to reduce green pigment adherence to the virus suggested that higher order charge organization was of importance, since the amine residues on the TMV 2663 virus surface are displayed in a regular array.

To mimic this charge organization, polyethylenimine (PEI) addition was evaluated. PEI is a highly branched polymer, one grade of which has a relatively high MW (˜70 kDa), with about 25% primary amine groups, 50% secondary amine groups and 25% tertiary amine groups. PEI is routinely employed in paper manufacturing for the neutralization of excess anionic colloidal charge. To function effectively, the solution pH is preferably below the pKa of the amine groups (neutral/acidic conditions), to ensure that the polymer is positively charged. These conditions were compatible with the pH 5 procedures above and PEI was evaluated at concentrations of 0.1% and 0.3% w/v, in the absence of heat treatment. PEI successfully partitioned the green pigments to the P1 at both polymer concentrations tested, resulting in a S1 supernatant with improved clarity. With PEG precipitation, the resuspended virus was white/cream in appearance in contrast with the green pigmentation obtained in the absence of PEI. These observations demonstrate that the positively charged polymer associates with host impurities, preferentially the anionic impurities, resulting in their aggregation and P1 partitioning. However, the composition profile by protein gel electrophoresis for the PEG precipitated pellets indicated that in the case of the 0.3% w/v PEI treatment, the principal protein was rubisco, with reduced levels of TMV 2663 coat protein detectable. At 0.1% PEI, a coat protein band was present, however rubisco impurities were substantially higher than for the control condition (no PEI). In contrast with the visual observations, gel electrophoresis appeared to contradict the hypothesis that the presence of the PEI polymer would promote TMV 2663 solubility.

PEI addition increased the GJ pH, to pH 6 and pH ˜7 for 0.1% w/v and 0.3% w/v PEI respectively, while the control was at pH 5.0 for the initial centrifugation to obtain the S1. Rubisco solubility increases above pH 5.2 and the higher pH may have also altered TMV 2663 solubility. To optimize the recovery of soluble virus and counteract the increase in soluble host protein impurities with PEI addition, studies with greater pH control were undertaken. Three levels of PEI (0.05, 0.1 and 0.3% w/v) were considered and for each PEI concentration, a portion of the GJ was readjusted to pH 5 prior to the initial clarification spin. At all PEI concentrations tested, the green pigment was effectively removed from the final virus. Virus recovery in the S1 was a function of both PEI concentration and pH. When readjustments to pH 5 were not performed, virus recovery decreased with increasing PEI concentration, from 27% at 0.05% w/v to 17% at 0.3% w/v. Conversely, with pH 5 readjustment, virus recovery increased with increasing PEI concentration, with 40% recovery in the S1 at 0.3% w/v PEI. For all PEI concentrations, the pH 5 readjustment was required for rubisco partitioning into the P1. These experiments suggested that with appropriate pH control, the incorporation of PEI into the extraction procedure was beneficial. Interestingly, the solubility characteristics of TMV 2663 differ from all other fusions analyzed to date in so far as solubility is decreased with increasing pH. A final set of experiments were performed, where PEI addition was combined with heat treatment and where the stage at which pH 5 readjustment occurred was considered. On the basis of final virus purity, the following procedure for processing the TMV 2663 fusion was selected:
(1) Extraction of infected tissue in two volumes of 0.86 M NaCl containing 0.04% w/v sodium metabisulfite.
(2) PEI addition to 0.3% w/v PEI with subsequent adjustment to pH 5.0.
(3) Heat treatment (47° C. for 5 minutes) with subsequent cooling to 10° C.
(4) Agitation at 4° C. for 20 minutes.
(5) Centrifugation at 6,000×g for 3 minutes to obtain the S1 supernatant.
(6) Adjustment to 4% w/v PEG 6,000, a 1 hour incubation at 4° C., and centrifugation at 10,000×g for 10 minutes to recover virus pellet.
(7) Repetition of PEG precipitation to obtain the final virus preparation.

This procedure was employed on the goat analog fusion G5(12) U1 (TMV U1827), with TMV 2663 processed in parallel. Extractions for both fusions, following the above procedure but omitting the PEI, were also performed. Visually, the PEI-based procedure successfully removed the green pigment from TMV U1827 yielding a virus preparation comparable in appearance to TMV 2663. Protein gel electrophoresis for the PEG precipitated virus demonstrated that the purity of the virus exceeded 90% for virus derived from the PEI procedure. Overall recovery for TMV U1827 was 60%, approximately double that of TMV 2663. The identity of each coat protein fusion was confirmed by mass spectrometry. Aggregation and settling of the purified virus was observed with storage. For TMV 2663, the final virus aggregated to a significantly greater extent when isolated by the PEI procedure. However, in the case of the TMV U1827 fusion, there was no marked difference in the extent of aggregation for the virus isolated with and without PEI treatment. This suggests that the increased precipitation of the 2663 virus was not due to the PEI procedure but rather was a characteristic of the virus following removal of the associated green pigment.

TABLE 26
Theoretical and experimentally observed molecular weights for the
DJ5(12) U1 (TMV 2663) and G5(12) U1 (TMV 1827) coat protein
fusions. The expected MW accounts for any post-translational
modifications to the coat protein fusion, which are listed.

| pLSB | Designation | Expected MW | Modifications | Observed MW |
|---|---|---|---|---|
| 2663 | DJ5(12) U1 | 18,793.94 Da | Met cleaved | 18,793.10 Da |
| | | [M + H] | | |
| 1827 | G5(12) U1 | 18,765.93 Da | Met cleaved | 18,764.77 Da |
| | | [M + H] | | |

A preliminary stability study was performed on the final virus and the virus in the S1 at pH 5. The latter condition represents a stringent test, since proteolytic activity is maximal in an acidic environment. Following storage for 5 days at 4° C., both coat protein fusions showed excellent stability, even at pH 5.

For the DJ5(20) U5 coat protein fusion (TMV 2659), the purification procedure involved contacting the S1 process stream with activated carbon, to adsorb host protein impurities. Recoveries were low and epitope truncation was observed during processing. Similar to the approach taken with the N-terminal U1 coat protein fusions, optimization was performed with the TMV 2659 construct, and the procedure developed then tested with G5(20) U5 (TMV U1826).

With TMV 2659, incorporation of NaCl during tissue homogenization did not increase virus partitioning into the S1; the majority of the virus remained associated with the P2, with approximately 10% recovered in each of the S1 and the S2. Similar to TMV 2663, comparisons between extraction with fresh and frozen tissue were performed and fresh tissue was chosen as minimal differences in virus yield and partitioning were observed. The 20 amino acid DJ5 peptide has an additional basic amino acid relative to the 12 amino acid coat protein fusion. Furthermore, two cysteines are present, introducing the possibility of oligomer formation by disulfide cross-linking between coat protein monomers, which was confirmed by non-reducing SDS-PAGE analysis. The incorporation of a reducing agent during homogenization was therefore tested with □-mercaptoethanol (□ME) added to the extraction buffer at a concentration of 100 mM. With NaCl present, virus partitioning into the S1 was improved substantially, and recoveries of 35-40% achieved. Approximately 25-30% of the virus remained in the P2 pellet, with the balance recovered in the S2.

The green pigmentation of the final virus was similar to TMV 2663. Host protein impurities were also unacceptably high for the PEG precipitated virus. As for TMV 2663, numerous unsuccessful studies were conducted to evaluate the use of bentonite during processing. Extraction in the presence of Tris was also tested. Although Tris failed to reduce final pigmentation or increase final purity, the partitioning characteristics of the TMV 2659 fusion were improved; the quantity of virus present in the S1 supernatant was doubled (65-70% recovery) with no virus remaining in the S2. During these initial experiments the PEG precipitation of TMV 2659 was suboptimal. Only 50% of the S1 TMV fusion was typically precipitated, compared to greater than 80% for TMV 2663 or its goat analog, with recoveries falling to 12% when Tris was incorporated into the buffer. This supported the observation that Tris improved TMV 2659 fusion solubility and also indicated that even with extraction in NaCl alone, 4% w/v PEG was insufficient. TMV 2659 employs a U5 backbone and studies with other fusions have indicated that higher PEG concentrations are sometimes required for aggregation of this capsid. Further testing indicated that near quantitative precipitation was obtained with 8% w/v PEG and that this level was also effective in cases where Tris was employed.

Owing to the improved recoveries observed with Tris, it was included as a variable during PEI testing. Extractions with or without PEI (0.3% w/v) and with or without Tris were compared. As for TMV 2663, the inclusion of PEI reduced green pigment levels in the S1, resulting in PEG precipitated virus that was tan/cream in appearance, while the virus pellets obtained in the absence of PEI were heavily pigmented. In contrast to TMV 2663, the 0.3% w/v PEI treatment was not detrimental to TMV 2659 extraction. With Tris present in addition to NaCl, virus levels in the S1 supernatant were unchanged at ˜70%, while for extraction in NaCl alone, PEI addition increased S1 recovery, from 30% to over 50%. Host protein impurities in the final virus preparation were not altered by PEI addition, with the level still requiring further reduction. To address this, extraction in the presence of PEI, with and without heat treatment, was considered with readjustment to pH 5.0 performed for a second set of process streams to compensate for the pH increase resulting from PEI addition. Virus partitioning and recovery were unaffected by the process modifications tested. From a purity standpoint, the pH 5 readjustment after PEI addition significantly improved P1 partitioning of rubisco in the absence of heat, while heat alone was also effective. However, the combination of heat and pH 5 readjustment produced the cleanest profile for the S1 supernatant and consequently for the PEG precipitated virus.

The susceptibility of the TMV 2659 fusion to proteolytic degradation was addressed. With short-term storage of the fusion at 4° C., a significant increase in the percentage of truncated species was evident by protein gel electrophoresis. A protease inhibitor screen was performed on green juice extracts to better characterize the proteolytic activity/activities responsible for this degradation. The plant tissue was homogenized in 0.86 M NaCl containing 100 mM BME and employed as is (pH 5.7) or adjusted to pH 5 and pH 8.5. Two protease inhibitor cocktails were employed, available commercially from Roche and Sigma. The composition of the Roche cocktail was not disclosed. Information regarding the Sigma protease cocktail components was provided, although the individual inhibitor concentrations were not indicated. In addition to the two cocktails, four other protease inhibitors were selected for the reasons listed below:

pAPMSF: Chosen as it is a specific and irreversible inhibitor for a class of serine proteases with a substrate specificity for positively charged amino acids. The DJ5 peptide contains one lysine and one arginine residue.

EACA: A reported carboxypeptidase inhibitor. No carboxypeptidase inhibitor was listed for the cocktails and the DJ5 peptide is located at the C-terminus of the coat protein.

Chymostatin: Inhibits several serine proteases and is also effective against lysosomal cysteine proteases. Previous work at LSBC had shown that chymostatin was effective against substilin-like proteases, which were identified as the predominant source of degradation for several recombinant TMV expressed proteins.

NEM (N ethylmaleimide): A cysteine protease inhibitor considered an economical alternative to E-64 or chymostatin.

The results of the screen strongly suggested that a cysteine protease with an acidic pH optimum was the principal activity responsible for proteolysis. No degradation was evident in the absence of protease inhibitors when the initial green juice was adjusted to pH 8.5 for storage, while truncation of the coat protein was detectable after 24 hours under acidic conditions (pH 5.7 and pH 5.0) and clearly visible by SDS-PAGE after 4 days of storage at 4° C. . From the 4-day storage data, the two inhibitor cocktails tested were of comparable effectiveness. Since the composition of the Sigma cocktail was known, further investigation was possible to define the inhibitor(s) responsible for maintaining the coat protein fusion integrity. Of the individual protease inhibitors tested, neither EACA nor pAPMSF were effective suggesting epitope location and basic residue content were not factors in degradation. No truncation occurred when the cysteine protease inhibitor NEM was employed, and chymostatin was also effective.

With TMV 2659, an extraction was performed in which either the Sigma cocktail or NEM were incorporated during processing, with inhibitor addition between the first and second PEG precipitations, and subsequent incubation for 1 hour at 4° C. Addition at this stage of processing was considered as reduced quantities of inhibitor were required owing to the volume reduction with PEG precipitation. Inhibitor concentration was also reduced by a factor of 10, relative to the levels employed in green juice, under the assumption that protease levels would be lower in the PEG precipitated virus compared to the starting GJ. After 5 days of storage at 4° C., degradation was observed for the PEG precipitated virus samples. In all cases, there were several truncation species migrating just below the putative full-length fusion and a ˜21 kDa band, although their relative intensities differed. The 21 kDa band was prominent for the virus purified by extraction in NaCl alone, while it was only a minor component for the virus recovered by extraction in Tris and was absent when the Tris-extracted virus was treated with NEM prior to the second PEG precipitation. Overall, the addition of the inhibitor(s) to the PEG precipitated virus did not appear to be as effective at preventing proteolysis as when the inhibitors were incorporated into the GJ. Higher concentrations of the inhibitors and/or different incubation conditions may be required. When analyzed by gel electrophoresis under non-reducing conditions, the characteristic multimeric banding pattern for the TMV 2659 was evident for all samples, indicating that disulfide cross-linking was present in the final virus. The gel also demonstrated that the final virus had attained an acceptable level of purity and the integrity of the epitope fusion in the initially purified virus was confirmed by mass spectrometry (Table 27).

TABLE 27
Theoretical and experimentally observed molecular weights for the
initially purified DJ5(20) U5 (TMV 2659) and G5(20) U5 (TMV
1826) coat protein fusions. The expected MW accounts for
any post-translational modifications to the coat protein
fusion, which are listed.

| | | | | Observed |
|---|---|---|---|---|
| pLSB | Designation | Expected MW | Modifications | MW |
| 2659 | DJ5(20) U5 | 19878.34 [M + H] | None | 19878.5 Da |
| 1826 | G5(20) U5 | 19850.32 [M + H] | None | 19856.57 Da |

This process, outlined below, was evaluated with the goat analog G5(20) U5 coat protein fusion (TMV U1826).
(1) Extraction of infected tissue in two volumes of 200 mM Tris, 0.86 M NaCl containing 100 mM □-mercaptoethanol, with pH 5.0 adjustment.
(2) Heat treatment (47° C. for 5 minutes) with subsequent cooling to 10° C.
(3) PEI addition to 0.3% w/v PEI with readjustment to pH 5.0.
(4) Agitation at 4° C. for 20 minutes.
(5) Centrifugation at 6,000×g for 3 minutes to obtain the S1 supernatant.
(6) Adjustment of S1 from pH 5 to pH 7.2 or pH 8.5 (to minimize pH 5 contact tme, since proteolysis is maximal under acidic conditions).
(7) Adjustment to 8% w/v PEG 6,000, a 1 hour incubation at 4° C., and centrifugation at 10,000×g for 10 minutes to recover virus pellet.
(8) Repetition of PEG precipitation to obtain final virus preparation.

Extraction in buffer lacking Tris was also performed, to determine if the incorporation of Tris improved fusion stability for TMV U1826 in a manner similar to that observed for TMV 2659. SDS-PAGE analysis , indicated that the freshly purified virus fusion migrated as a single distinct band under reducing conditions and identity was confirmed by mass spectrometry (Table 27). From the standpoint of recovery, this experiment indicated that the incorporation of Tris did not improve S1 partitioning for the U5 goat analog, with a 50/50 split between the S1 and the P1 observed in both cases, resulting in a final recovery of 40%. With storage for 5 days at 4° C., degradation was observed in all cases, however, the presence of Tris did reduce the extent of truncation. This confirmed that Tris was an important component of the extraction buffer in the case of the U5 coat protein fusions. The pH to which the S1 was adjusted (7.2 or 8.5) also influenced stability, with the higher pH being preferential. In summary, success has been obtained with the inclusion of protease inhibitors and Tris during extraction of the DJ5(20) and G5(20) U5 coat protein fusions, together with pH control during processing.

### 7. INCORPORATION OF REFERENCES

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the claims. Various publications are cited herein, the disclosures of which are incorporated by reference in their entireties.

LIST OF SEQ IDs

| SEQ | |
|---|---|
| ID | Nucleic acid sequence |
| 1S | 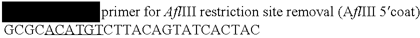 |
| 2S | 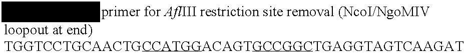 |
| 3S | 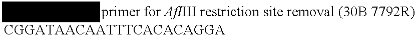 |
| 4S | 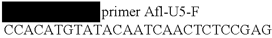 |
| 5S | 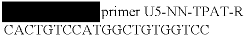 |
| 6S | Oligonucleotide primer U5-NN-TPAT |
| | CTTGTCTGGACCACAGCCATGGACAGTGCCGGCACTCCGGCTACTT |
| | AG |
| 7S | Oligonucleotide primer JAL302 |
| | AAACATGATTACGCCAAGCTTGCATG |
| 8S | Peptide acceptor vector pLSB2110 open reading frame |
| | ATGTCTTACAGTATCACTACTCCATCTCAGTTCGTGTTCTTGTCATC |
| | AGCGTGGGCCGACCCAATAGAGTTAATTAATTTATGTACTAATGCC |
| | TTAGGAAATCAGTTTCAAACACAACAAGCTCGAACTGTCGTTCAAA |
| | GACAATTCAGTGAGGTGTGGAAACCTTCACCACAAGTAACT |
| | GTTAGGTTCCCTGACAGTGACTTTAAGGTGTACAGGTACAATGCGG |
| | TATTAGACCCGCTAGTCACAGCACTGTTAGGTGCATTCGACACTAG |
| | AAATAGAATAATAGAAGTTGAAAATCAGGCGAACCCCACGACTGC |
| | CGAGACGTTAGATGCTACTCGTAGAGTAGACGACGCAACGGTG |
| | GCCATAAGGAGCGCGATAAATAATTTAATAGTAGAATTGATCAGA |
| | GGAACCGGATCTTATAATCGGAGCTCTTTCGAGAGCTCTTCTGGTTT |
| | GGTTTGGACCTCTGGTCCTGCAACTGCCATGGACAGTGCCGGCTGA |
| 9S | Peptide acceptor vector pLSB1806 open reading frame |
| | ATGTATACAATCAACTCTCCGAGCCAATTTGTTTACTTAAGTTCCGC |
| | TTATGCAGATCCTGTGCAGCTGATCAATCTGTGTACAAATGCATTG |
| | GGTAACCAGTTTCAAACGCAACAAGCTAGGACAACAGTCCAACAG |
| | CAATTTGCGGATGCCTGGAAACCTGTGCCTAGTATGACAGTG |
| | AGATTTCCTGCATCGGATTTCTATGTGTATAGATATAATTCGACGCT |
| | TGATCCGTTGATCACGGCGTTATTAAATAGCTTCGATACTAGAAAT |
| | AGAATAATAGAGGTTGATAATCAACCCGCACCGAATACTACTGAA |
| | ATCGTTAACGCGACTCAGAGGGTAGACGATGCGACTGTAGCT |
| | ATAAGGGCTTCAATCAATAATTTGGCTAATGAACTGGTTCGTGGAA |
| | CTGGCATGTTCAATCAAGCAAGCTTTGAGACTGCTAGTGGACTTGT |
| | CTGGACCACAGCCATGGACAGTGCCGGCACTCCGGCTACTTAG |
| 10S | 20 amino acid DJ5 epitope |
| | VHQANPRGSAGPCCTPTKMS |
| 11S | 12 amino acid DJ5-derived epitope |
| | VHQANPRGSAGP |
| 12S | Forward oligonucleotide for 20 amino acid DJ5 peptide cloning |
| | CATGGTTCATCAACGTAATCCAAGAGGATCTGCTGGACCATGTTGT |
| | ACTCCAACTAAGATGTCTG |
| 13S | Reverse oligonucleotide for 20 amino acid DJ5 peptide cloning |
| | CCGGCAGACATCTTAGTTGGAGTACAACATGGTCCAGCAGATCCTC |
| | TTGGATTAGCTTGATGAAC |
| 14S | ORF for vector pLSB2655 |
| | ATGTCTTACAGTATCACTACTCCATCTCAGTTCGTGTTCTTGTCATC |
| | AGCGTGGGCCGACCCAATAGAGTTAATTAATTTATGTACTAATGCC |
| | TTAGGAAATCAGTTTCAAACACAACAAGCTCGAACTGTCGTTCAAA |
| | GACAATTCAGTGAGGTGTGGAAACCTTCACCACAAGTAACTGTTAG |
| | GTTCCCTGACAGTGACTTTAAGGTGTACAGGTACAATGCGGTATTA |
| | CACCCGCTAGTCACAGCACTGTTAGGTGCATTCGACACTAGAAATA |
| | GAATAATAGAAGTTGAAAATCAGGCGAACCCCACGACTGCCGAGA |
| | CGTTAGATGCTACTCGTAGAGTAGACGACGCAACGGTGGCCATAA |
| | GGAGCGCGATAAATAATTTAATAGTAGAATTGATCAGAGGAACCG |
| | GATCTTATAATCGGAGCTCTTTCGAGAGCTCTTCTGGTTTGGTTTGG |
| | ACCTCTGCCATGGTTCATCAAGCTAATCCAAGAGGATCTGCTGGAC |
| | CATGTTGTACTCCAACTAAGATGTCTGCCGGCCCTGCAACTTGA |
| 15S | ORF for vector pLSB2656 |
| | ATGTCTTACAGTATCACTACTCCATCTCAGTTCGTGTTCTTGTCATC |
| | AGCGTGGGCCGACCCAATAGAGTTAATTAATTTATGTACTAATGCC |
| | TTAGGAAATCAGTTTCAAACACAACAAGCTCGAACTGTCGTTCAAA |
| | GACAATTCAGTGAGGTGTGGAAACCTTCACCACAAGTAACTGTTAG |
| | GTTCCCTGACAGTGACTTTAAGGTGTACAGGTACAATGCGGTATTA |
| | GACCCGCTAGTCACAGCACTGTTAGGTGCATTCGACATAGAAATAG |
| | AATAATAGAAGTTGAAAATCAGGCGAACCCCACGACTGCCGAGACG |
| | TTAGATGCTACTCGTAGAGTAGACGACGCAACGGTGGCCATAAGGA |
| | GCGCGATAAATAATTTAATAGTAGAATTGATCAGAGGAACCGGATC |
| | TTATAATCGGAGCTCTTTCGAGAGCTCTTCTGGTTTGGTTTGGACC |
| | TCTGGTCCTGCAACTGCCATGGTTCATCAAGCTAATCCAAGAGGAT |
| | CTGCTGGACCATGTTGTACTCCAACTAAGATGTCTGCCGGCTGA |
| 16S | ORF for vector pLSB2657 |
| | ATGGTTCATCAAGCTAATCCAAGAGGATCTGCTGGACCATGTTGTA |
| | CTCCAACTAAGATGTCTGCCGGCTCTTACAGTATCACTACTCCATCT |
| | CAGTTCGTGTTCTTGTCATCAGCGTGGGCCGACCCAATAGAGTTAA |
| | TTAATTTATGTACTAATGCCTTAGGAAATCAGTTTCAAACACAACA |
| | AGCTCGAACTGTCGTTCAAAGACAATTCAGTGAGGTGTGGAAACCT |
| | TCACCACAAGTAACTGTTAGGTTCCCTGACAGTGACTTTAAGGTGT |
| | ACAGGTACAATGCGGTATTAGACCCGCTAGTCACAGCACTGTTAGG |
| | TGCATTCGACACTAGAAATAGAATAATAGAAGTTGAAAATCAGGC |
| | GAACCCCACGACTGCCGAGACGTTAGATGCTACTCGTAGAGTAGAC |
| | GACGCAACGGTGGCCATAAGGAGCGCGATAAATAATTTAATAGTA |
| | GAATTGATCAGAGGAACCGGATCTTATAATCGGAGCTCTTTCGAGA |
| | GCTCTTCTGGTTTGGTTTGGACCTCTGGTCCTGCAACTTGA |
| 17S | ORF for vector pLSB2658 |
| | ATGTCTTACAGTATCACTACTCCATCTCAGTTCGTGTTCTTGTCATC |
| | AGCGTGGGCCGACCCAATAGAGTTAATTAATTTATGTACTAATGCC |
| | TTAGGAAATCAGTTTCAAACACAACAAGCTCGAACTGTCGTTCAAA |
| | GACAATTCAGTGAGGTGTGGAAACCTTCACCACAAGTAACTGTTAG |
| | GTTCCCTGGATCTCCCATGGTTCATCAAGCTAATCCAAGAGGATCT |
| | GCTGGACCATGTTGTACTCCAACTAAGATGTCTGCCGGCCCTTCTG |
| | GAGACTTTAAGGTATACAGGTACAATGCGGTATTAGACCCGCTAGT |
| | CACAGCACTGTTAGGTGCATTCGACACTAGAAATAGAATAATAGA |
| | AGTTGAAAATCAGGCGAACCCCACGACTGCCGAGACGTTAGATGC |
| | TACTCGTAGAGTAGACGACGCAACGGTGGCCATAAGGAGCGCGAT |
| | AAATAATTTAATAGTAGAATTGATCAGAGGAACCGGATCTTATAAT |
| | CGGACGTCTTTCGAGAGCTCTTCTGGTTTGGTTTGGACCTGTGGTCC |
| | TGCAACTTGA |
| 18S | ORF for vector pLSB2659 |
| | ATGTATACAATCAACTCTCCGAGCCAATTTGTTTACTTAAGTTCCGC |
| | TTATGCAGATCCTGTGCAGCTGATCAATCTGTGTACAAATGCATTG |
| | GGTAACCAGTTTCAAACGCAACAAGCTAGGACAACAGTCCAACAG |
| | CAATTTGCGGATGCCTGGAAACCTGTGCCTAGTATGACAGTGAGAT |
| | TTCCTGCATCGGATTTCTATGTGTATAGATATAATTCGACGCTTGAT |
| | CCGTTCATCACGGCGTTATTAAATAGCTTCGATACTAGAAATAGAA |
| | TAATAGAGGTTGATAATCAACCCGCACCGAATACTACTGAAATCGT |
| | TAACGCGACTCAGAGGGTAGACGATGCGACTGTACGCTATAAGGGC |
| | TTCAATCAATAATTTGGCTAATGAACTGGTTCGTGGAACTGGCATG |
| | TTCAATCAAGCAAGCTTTGAGACTGCTAGTGGACTTGTCTGGACCA |
| | CAGCCATGGTTCATCAAGCTAATCCAAGAGGATCTGCTGGACCATG |
| | TTGTACTCCAACTAAGATGTCTGCCGGCACTCCGGCTACTTAG |
| 19S | Coat protein amino acid sequence obtained from pLSB2655 |
| | MSYSITTPSQFVFLSSAWADPIELINLCTNALGNQFQTQQARTVVQRQF |
| | SEVWKPSPQVTVRFPDSDFKVYRYNAVLDPLVTALLGAFDTRNRIIEV |
| | ENQANPTTAETLDATRRVDDATVAIRSAINNLIVELIRGTGSYNRSSFE |
| | SSSGLVWTSAMVHQANPRGSAGPCCTPTKMSAGPAT |
| 20S | Coat protein amino acid sequence obtained from pLSB2656 |
| | SYSITTPSQFVFLSSAWADPIELINLCTNALGNQFQTQQARTVVQRQFS |
| | EVWKPSPQVTVRFPDSDFKVYRYNAVLDPLVTALLGAFDTRNRIIEVE |
| | NQANPTTAETLDATRRVDDATVAIRSAINNLIVELIRGYGSYNRSSFESS |
| | SGLVWTSGPATAMVHQANPRGSAGPCCTPTKMSAG |
| 21S | Coat protein amino acid sequence obtained from pLSB2657 |
| | MVHQANPRGSAGPCCTPTKMSAGSYSITTPSQFVFLSSAWADPIELINL |
| | CTNALGNQFQTQQARTVVQRQFSEVWKPSPQVTVRFPDSDFKVYRYN |
| | AVLDPLVTALLGAFDTRNRIIEVENQANPTTAETLDATRRVDDATVAI |
| | RSAINNLIVELIRGTGSYNRSSFESSSGLVWTSGPAT |
| 22S | Coat protein amino acid sequence obtained from pLSB2658 |
| | MSYSITTPSQFVFLSSAWADPIELINLCTNALGNQFQTQQARTVVQRQF |
| | SEVWKPSPQVTVRFPGSPMVHQANPRGSAGPCCTPTKMSAGPSGDFK |
| | VYRYNAVLDPLVTALLGAFDTRNRIIEVENQANPTTAETLDATRRVDD |
| | ATVAIRSAINNLIVELIRGTGSYNRSSFESSSGLVWTSGPAT |
| 23S | Coat protein amino acid sequence obtained from pLSB2659 |
| | MYTINSPSQFVYLSSAYADPVQLINLCTNALGNQFQTQQARTTVQQQF |
| | ADAWKPVPSMTVRFPASDFYVYRYNSTLDPLITALLNSFDTRNRIIEVD |
| | NQPAPNTTEIVNATQRVDDATVAIRASINNLANELVRGTGMFNQASFE |
| | TASGLVWTTAMVHQANPRGSAGPCCTPTKMSAGTPAT |
| 24S | 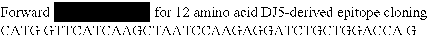 |
| 25S | 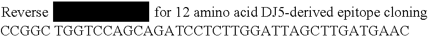 |
| 26S | ORF for vector pLSB2663 |
| | ATGGTTCATCAAGCTAATCCAAGAGGATCTGCTGGACCAGCCGGCT |
| | CTTACAGTATCACTACTCCATCTCAGTTCGTGTTCTTGTCATCAGCG |
| | TGGGCCGACCCAATAGAGTTAATTAATTTATGTACTAATGCCTTAG |
| | GAAATCAGTTTCAAACACAACAAGCTCGAACTGTCGTTCAAAGACA |
| | ATTCAGTGACCTGTGGAAACCTTCACCACAAGTAACTGTTAGGTTC |
| | CCTGACAGTGACTTTAAGGTGTACAGGTACAATGCGGTATTAGACC |
| | CGCTAGTCACAGCACTGTTAGGTGCATTCGACACTAGAAATAGAAT |
| | AATAGAAGTTGAAAATCAGGCGAACCCCACGACTGCCGAGACGTT |
| | AGATGCTACTCGTAGAGTAGACGACGCAACGGTGGCCATAAGGAG |
| | CGCGATAAATAATTTAATAGTAGAATTGATCAGAGGAACCGGATCT |
| | TATAATCGGAGCTCTTTCGAGAGCTCTTCTGGTTTGGTTTGGACCTC |
| | TGGTCCTGCAACTTGA |
| 27S | ORF for vector pLSB2664 |
| | ATGTCTTACAGTATCACTACTCCATCTCAGTTCGTGTTCTTGTCATC |
| | AGCGTGGGCCGACCCAATAGAGTTAATTAATTTATGTACTAATGCC |
| | TTAGGAAATCAGTTTCAAACACAACAAGCTCGAACTGTCGTTCAAA |
| | GACAATTCAGTGAGGTGTGGAAACCTTCACCACAAGTAACTGTTAG |
| | GTTCCCTGACAGTGACTTTAAGGTGTACAGGTACAATGCGGTATTA |
| | GACCCGCTAGTCACAGCACTGTTAGGTGCATTCGACACTAGAAATA |
| | GAATAATAGAAGTTGAAATCAGGCGAACCCCACGACTGCCGAGA |
| | CGTTAGATGCTACTCGTAGAGTAGACGACGCAACGGTGGCCATAA |
| | GGAGCGCGATAAATAATTTAATAGTAGAATTGATCAGAGGAACCG |
| | GATCTTATAATCGGAGCTCTTTCGAGAGCTCTTCTGGTTTGGTTTGG |
| | ACCTCTGCCATGGTTCATCAAGCTAATCCAAGAGGATCTGCTGGAC |
| | CAGCCGGCCCTGCAACTTGA |
| 28S | Coat protein amino acid sequence obtained from pLSB2663 |
| | MVHQANPRGSAGPAGSYSITTPSQFVFLSSAWADPIELINLCTNALGNQ |
| | FQTQQARTVVQRQFSEVWKPSPQVTVRFPDSDFKVYRYNAVLDPLVT |
| | ALLGAFDTRNRIIEVENQANPTTAETLDATRRVDDATVAIRSAINNLIV |
| | ELIRGTGSYNRSSFESSSGLVWTSGPAT |
| 29S | Coat protein amino acid sequence obtained from pLSB2664 |
| | MSYSITTPSQFVFLSSAWADPIELINLCTNALGNQFQTQQARTVVQRQF |
| | SEVWKPSPQVTVRFPDSDFKVYRYNAVLDPLVTALLGAFDTRNRIIEV |
| | ENQANPTTAETLDATRRVDDATVAIRSAINNLIVELIRGYGSYNRSSFE |
| | SSSGLVWTSAMVHQANPRGSAGPAGPAT |
| 30S | Wild-type TMV strain U1 coat protein |
| | MSYSITTPSQFVFLSSAWADPIELINLCTNALGNQFQTQQARTVVQRQF |
| | SEVWKPSPQVTVRFPDSDFKVYRYNAVLDPLTALLGAFDTRNRIIEV |
| | ENQANPTTAETLDATRRVDDATVAIRSAINNLIVELIRGTGSYNRSSFE |
| | SSSGLVWTSGPAT |
| 31S | Wild-type TMV strain U5 coat protein |
| | MPYTINSPSQFVYLSSAYADPVQLINLCTNALGNQFQTQQARTTVQQQ |
| | FADAWKPVPSMTVRFPASDFYVYRYNSTLDPLITALLNSFDTRNRIIEV |
| | DNQPAPNTTEIVNATQRVDDATVAIRASINNLANELVRGTGMFNQASF |
| | ETASGLVWTTTPAT |

1. A method for preventing protein degradation while extracting or storing a virus or a protein from primary biological material comprising;
purifying or storing the virus or protein at a pH greater than about 7.

2. The method of claim 1 wherein the pH is greater than 7.5.

3. The method of claim 1 further comprising recovering a purified virus.

4. The method of claim 3 wherein the virus contains a coat protein fused to an antigenic epitope.

5. The method of claim 1 wherein the protein is under expression control by the virus and the protein is not native to wild-type virus.

6. A method for preventing protein or nucleic acid degradation while extracting or storing a virus, a nucleic acid or a protein containing material from biological material comprising; adding activated carbon or other protease/nuclease adsorbent to a biological extract, allowing the protease/nuclease to adsorb to the activated carbon or other protease/nuclease adsorbent, and removing the activated carbon or other protease/nuclease adsorbent from the extract.

7. The method of claim 6 further comprising recovering a purified virus.

8. The method of claim 7 wherein the virus contains a coat protein fused to an antigenic epitope.

9. The method of claim 6 wherein the protein is under expression control by the virus and the protein is not native to wild-type virus.

10. A method for solubilizing virus or virus proteins comprising increasing the ionic strength or adding sufficient salt to prevent aggregation of virus or virus proteins.

11. The method of claim 10 wherein the virus contains a coat protein fused to an antigenic epitope.

12. The method of claim 10 the virus protein is under expression control by the virus and the protein is not native to wild-type virus.

13. The method of claim 6 wherein a protein or VLP or a nucleic acid is recovered.
